SEQUENCE LISTING (40 sequences)

SEQ ID NO: 1 - protein, 296 aa, Campylobacter hepaticus
MRKAIIAGNGPSLKEIDYTKLPIDYDVFRCNQFYFEDKYYLGKNCKAVFYNPSLFFEQYYTLKHLIDKKEYKTDFIFCSTFNLVHLENENFSKIFYNYFPDAHLGYDFLKTLKEFDAYCKFHEIYLNQRITSGIYMCAIAIALGYKEIYLAGIDFYHNGSFYAFNTKQNNLIKLLPNFKNDNSHNIKHTKNMDIKALEFLEKTYEVQFYCLCPNSPLSHFIKTPPPVKNSTFKLEEKSNYIKDILIPSKEAYDIFSINFNVSKKPRLKQNIYYRLIENLLKLPSDIKHYYKSRKLK

SEQ ID NO: 2 - protein, 326 aa, Haemophilus influenzae
MNGTICPINQSINQSINQSINQSINQSINQSKSVIIAGNGTSLKSIDYSLLPKDYDVFRCNQFYFEDHYFLGKKIKKVFFNCSVIFEQYYTFMQLIKNNEYEYADIILSSFLNLGDSELKKIQRLEKLLPQIDLGHSYLKKLRAFDAHLQYHELYENKRITSGVYMCAVATAMGYKDLYLTGIDFYQEKGNPYAFHHQTENIIKLLPSFSQNKSQSDIHSMEYDLNALYFLQKHYGVNIYCISPESPLCNYFPLSPLNNPITFILEEKKNYTQDILIPPKFVYKKIGIYSKPRIYQNLIFRLIWDILRLPNDIKHALKAKKMRLRK

SEQ ID NO: 3 - protein, 292 aa, Campylobacter coli
MKKVIIAGNGPSLKEIDYSRLPNDFDVFRCNQFYFEDKYYLGKKCKAVFYNPSLFFEQYYTLKHLIQNQEYETELIVCSNFNLTHIESENFLKNFYDYFPDAHLGYDFFKQLKEFNAYFKFHEIYFNQRITSGIYMCAVAIALGYKEIYLSGIDFYQNGSSYAFDTKQKNLLKLVSNFKNDNSHYIGHSKNTDLKALEFLEKTYEIKLYCLCPNSLLANFIELAPNLNSNFIIQEKNNYIKDILIPSKEAYNNFSKNFNLPNKPKLKENIYFKILQDLLKLPSDIKHYFKGK

SEQ ID NO: 4 - protein, 289 aa, Campylobacter jejuni
MKKAIIAGNAPSLKNIDYTLLPQDYDVFRCNQFYLEDKYYLGKKLKAVFFNSCVFFENYYTLKELIKNEEYTTSLIFCSSHKHLEEKDFLENFKDFYPDSTMGHEILSRLEKFYAWTIFNDVYKNRRFTSAIYMCAIAVAMGYKELYLAGIDFYNTGSTYAYTQGENLTRIFGHFKKYNGHTQDIELEALELLKELYDVKIYCLCPTSPLAKFVTPPPSTSHLNHFILENKPQNHINDMLLPSKRAYKNARKLRNYDNSKLTDNLYYRLIKDLIKLPSNIKHHLKTRRF

SEQ ID NO: 5 - protein, 392 aa, Pasteurella oralis
MSNWAKSVTIYLDPASLPALNQLMDFTQKSDDKETARIFGHTRFKMPEEIQKQYQHIDMVGIKDEKPTEELFSILDQYPESLELDLHMSIAHATKLIQPILAYRFKHPSRVSIRSLNLYDDGSLEYVGLENLQDVDIPKAIAQAEQQLASFLMTGKAKFDNPIVARYVWQSQFPVKYHFLSPEYFEKAAFIKPLKEYLQDNYQKMALFAYQDLSSEKQALYLKLVGFNDQIKQLLETTEKKFIFTGTTTWEAKTDVREYYAQQQLNLLKHFTQPNGELFIGDDYKVYFKGHPKGDEINEYILNNAKDIINIPANISFEILMMTGLLPDKVGGIASSLYFSLPKEKISHIIFTTNKQVKSKEDALNNPYVKVMQRLGIIDESQVIFWDTLKQL

SEQ ID NO: 6 - protein, 259 aa, Campylobacter jejuni
MKKVIIAGNGPSLKEIDYSRLPNDFDVFRCNQFYFEDKYYLGKKCKAVFYNPILFFEQYYTLKHLIQNQEYETELIMCSNYNQAHLENENFVKTFYDYFPDAHLGYDFFKQLKDFNAYFKFHEIYFNQRITSGVYMCAVAIALGYKEIYLSGIDFYQNGSSYAFDTKQKNLLKLAPNFKNDNSHYIGHSKNTDIKALEFLEKTYKIKLYCLCPNSLLANFIELAPNLNSNFIIQEKNNYTKDILIPSSEAYGKFSKNIN

SEQ ID NO: 7 - DNA, 891 bp, Campylobacter hepaticus
  source 1..891: mol_type = other DNA
atgcgtaaagcaattattgcaggtaatggtccgagcctgaaagaaatcgattataccaaactgccgatcgattatgatgtgtttcgttgcaaccagttctatttcgaggataaatactacctgggcaaaaattgcaaagccgtgttttataacccgagcctgtttttcgaacagtactataccctgaaacacctgatcgacaaaaaagagtataagaccgatttcatcttctgcagcacctttaatctggtgcatctggaaaatgagaactttagcaagatcttctacaactattttccggatgctcatctgggctatgattttctgaaaacgctgaaagaatttgatgcctactgcaaattccacgagatttatctgaatcagcgtattaccagcggcatttatatgtgtgcaattgccattgcgctgggctataaagaaatttatctggcaggcatcgacttctatcataacggtagcttttatgccttcaacaccaaacagaacaacctgattaaactgctgccgaacttcaaaaacgataacagccataacatcaaacacaccaagaacatggatatcaaagccctggaatttctggaaaaaacctatgaggttcagttctactgtctgtgtccgaatagtccgctgagccatttcattaaaacaccgcctccggtgaaaaatagcacgtttaaactggaagagaaaagcaactacatcaaagacattctgatcccgagcaaagaagcctacgatatctttagcattaactttaacgtgagcaagaaaccgcgtctgaaacagaatatctattatcgcctgattgagaacctgctgaaactgccgtcagatatcaaacactattacaaaagccgcaaactgaaataa

SEQ ID NO: 8 - DNA, 981 bp, Haemophilus influenzae
  source 1..981: mol_type = other DNA
atgaacggcaccatttgtccgattaatcagagcattaaccagtccatcaaccagagcatcaatcaatccattaatcagtcaattaatcaaagcaaaagcgtgatcattgcaggtaatggcaccagcctgaaaagcattgattatagcctgctgccgaaagattatgatgtgtttcgttgcaaccagttctatttcgaggatcattattttctgggcaaaaagatcaaaaaggtgttctttaactgcagcgtgatcttcgaacagtattatacctttatgcagctgatcaaaaacaacgagtatgagtatgccgatattatcctgagcagctttctgaatctgggtgatagcgaactgaaaaagattcagcgtctggaaaaactgctgccgcagattgatctgggtcatagttatctgaagaaactgcgtgcatttgatgcacatctgcagtatcatgaactgtatgaaaacaaacgtattaccagcggtgtttatatgtgtgcagttgcaaccgcaatgggttataaagatctgtatctgaccggcatcgatttctatcaagaaaaaggtaatccgtatgcctttcatcatcagaccgaaaacattatcaaactgttaccgagcttcagccagaataaaagccagagcgatattcacagcatggaatatgatctgaacgccctgtatttcctgcagaaacattatggcgttaacatctattgcatcagtccggaaagtccgctgtgtaactattttccgctgagtccgctgaataatccgattaccttcattctggaagagaaaaagaactacacccaggatattctgatccctccgaaattcgtgtataaaaagattggcatctatagcaagcctcgcatttatcagaatctgatttttcgcctgatttgggatatcctgcgtctgccgaatgatattaaacatgcactgaaagccaaaaagatgcgcctgcgtaaataa

SEQ ID NO: 9 - DNA, 879 bp, Campylobacter coli
  source 1..879: mol_type = other DNA
atgaagaaggtgattattgcaggtaatggtccgagcctgaaagaaattgattatagccgtctgccgaacgattttgatgtttttcgttgcaaccagttctacttcgaggataaatactatctgggcaaaaagtgcaaagccgtgttttataacccgagcctgtttttcgaacagtactataccctgaaacatctgatccagaatcaagagtatgaaaccgaactgattgtgtgcagcaactttaatctgacccatatcgaaagcgagaacttcctgaaaaacttctatgattattttccggatgcgcacctgggctatgatttctttaaacagctgaaagagttcaacgcctacttcaaatttcacgagatctattttaaccagcgtattaccagcggcatttatatgtgtgcagttgcaattgcgctgggctataaagaaatttatctgagcggcatcgatttctatcagaatggtagcagctatgcctttgacaccaaacagaaaaatctgctgaaactggtgagcaacttcaaaaacgataacagccattatatcggccacagcaaaaataccgatctgaaagcactggaatttctggaaaagacctatgagatcaaactgtattgtctgtgtccgaatagcctgctggcaaactttattgaactggcaccgaatctgaactccaactttattatccaagagaaaaacaactacatcaaagatattctgatcccgagcaaagaggcctataacaactttagcaaaaacttcaacctgccgaacaaaccgaaactgaaagagaacatctatttcaaaattctgcaggacctgctgaaattgccgagcgatatcaaacattacttcaagggcaaataa

SEQ ID NO: 10 - DNA, 870 bp, Campylobacter jejuni
  source 1..870: mol_type = other DNA
atgaagaaagccattattgcaggtaatgcaccgagcctgaaaaacattgattataccctgctgccgcaggattatgatgtttttcgttgcaatcagttctatctggaagataaatactacctgggcaaaaagctgaaagccgtgtttttcaatagctgcgtgttcttcgaaaactattacaccctgaaagagctgatcaagaacgaagaatataccacgagcctgatcttttgtagcagccataaacacctggaagagaaagattttctggaaaacttcaaagacttctatccggattcaaccatgggtcatgaaattctgagccgtctggaaaaattctatgcatggaccattttcaacgatgtgtataaaaaccgtcgttttaccagcgccatttatatgtgtgcaattgccgttgcaatgggctataaagaactgtatctggcaggcatcgatttctataataccggtagcacctatgcatatacccagggtgaaaatctgacccgtatttttggtcacttcaagaaatataacggccacacgcaggatattgaactggaagcactggaactgctgaaagaattatatgacgtgaagatctattgtctgtgtccgaccagtccgctggcaaaatttgttacaccgcctccgagcaccagccatctgaatcattttatcctggaaaataaaccgcagaaccacatcaatgatatgctgctgccgagcaaacgtgcctataaaaacgcacgtaaactgcgcaattatgacaatagcaaactgaccgataacctgtattatcgcctgatcaaagacctgattaaactgccgagtaacatcaaacatcatctgaaaacccgtcgcttctaa

SEQ ID NO: 11 - DNA, 1179 bp, Pasteurella oralis
  source 1..1179: mol_type = other DNA
atgagcaattgggcaaaaagcgtgaccatttatctggatccggcaagcctgcctgcactgaaccagctgatggattttacccagaaatccgatgataaagaaaccgcacgtatttttggtcacacccgctttaaaatgccggaagaaattcagaaacagtaccagcatattgatatggtgggcatcaaagatgaaaaaccgaccgaagaactgtttagcattctggatcagtatccggaaagtctggaactggatctgcatatgagcattgcacatgcaaccaaactgattcagccgattctggcatatcgttttaaacatccgagccgtgttagcattcgtagcctgaatctgtatgatgatggtagcctggaatatgttggtctggaaaatctgcaggatgtggatattccgaaagcaattgcacaggcagaacagcagctggcaagctttctgatgaccggtaaagcaaaatttgataatccgattgttgcccgttatgtttggcagagccagtttccggttaaatatcattttctgagtccggaatatttcgagaaagcagcatttatcaaaccgctgaaagaatacctgcaggacaattaccagaaaatggcactgtttgcatatcaggatctgagcagcgaaaaacaggcactgtatctgaaactggtgggttttaacgatcagattaaacagctgctggaaaccaccgagaaaaagtttatctttaccggcaccaccacctgggaagcaaaaaccgatgttcgtgaatattatgcacagcagcagctgaatctgctgaaacattttacacagccgaatggcgaactgttcattggtgatgattacaaggtgtactttaaaggccatccgaaaggcgacgaaatcaatgaatatatcctgaacaacgccaaagacatcattaacattccggcaaacatcagctttgaaattctgatgatgacaggtctgctgccggataaagttggtggtattgcaagcagcctgtattttagcctgccgaaagagaaaattagccacatcattttcaccaccaacaagcaggttaaaagcaaagaagatgcactgaataacccgtacgttaaagttatgcagcgtctgggtattattgatgaaagccaggttatcttttgggacaccctgaaacagctgtaa

SEQ ID NO: 12 - DNA, 780 bp, Campylobacter jejuni
  source 1..780: mol_type = other DNA
atgaagaaagttattattgcaggtaatggtccgagcctgaaagaaattgattatagccgtctgccgaacgattttgatgtttttcgttgcaaccagttctacttcgaggataaatactacctgggcaaaaaatgcaaagccgtgttttataacccgatcctgtttttcgaacagtactataccctgaaacatctgatccagaatcaagagtatgaaaccgaactgattatgtgcagcaattataaccaggcgcatctggaaaatgagaactttgtgaaaaccttctatgactattttccggatgcacatctgggctacgattttttcaaacagctgaaagatttcaacgcctacttcaaatttcacgagatctatttcaatcagcgcattaccagcggtgtttatatgtgtgcagttgcaattgccctgggctataaagaaatttatctgagcggcatcgatttctatcagaatggtagcagctatgcctttgacaccaaacagaaaaatctgctgaaactggcaccgaacttcaaaaatgataacagccattatatcggccacagcaaaaacaccgatattaaagcactggaatttctggaaaaaacctataaaatcaaactgtattgtctgtgcccgaatagcctgctggcaaactttattgaattagcaccgaatctgaacagcaacttcatcatccaagagaagaacaactacaccaaggatattctgattccgagcagcgaagcatatggcaaatttagcaaaaatatcaattaa

SEQ ID NO: 13 - DNA, 2818 bp, Campylobacter jejuni
  source 1..2818: mol_type = other DNA
atgaaagaaataaaaatacaaaatataatcataagtgaagaaaaagcacccttagtcgtacctgaaataggcattaatcataatggcagtttagaactagctaaaattatggtagatgcagcctttagcgcaggtgctaagattataaagcatcaaactcatattgttgaagatgagatgagtaaggccgctaaaaaagtaattcctggtaatgcaaaaataagcatttatgagattatgcaaaaatgtgctttggattataaagatgagctagcacttaaagaatacacagaaaaattaggtcttgtttatcttagcacacctttttctcgtgcaggtgcgaaccgcttagaagatatgggagttagtgcttttaagattggttcaggtgagtgtaataattatccgcttattaaacacatagcagcctttaaaaagcctatgatagttagcacaggaatgaatagtattgaaagtataaaaccaactgtaaaaatcttattagacaatgaaattccttttgttttaatgcacacgaccaatctttacccaaccccgcataatcttgtaagattaaacgctatgcttgagttaaaaaaagaattttcttgtatggtaggcttaagcgaccacacaacagataatcttgcgtgtttaggtgcagttgtacttggagcttgtgtgcttgaaagacattttactgatagtatgcatagaagtggccctgatatagtttgttctatggatacaaaggctttaaaagagctaattatacaaagtgagcaaatggctataataagaggaaataatgaaagtaaaaaagcggctaaacaagaacaagttacaattgattttgcctttgcaagtgtagttagcattaaagatattaaaaaaggcgaagttttatctatggataatatttgggttaaaagacctggacttggtggaattagtgcggctgaatttgaaaatattttaggcaaaaaagcattaagagatatagaaaatgatgctcagttaagctatgaggattttgcgtgaaaaaaatcctttttataacaggctctagggctgattattctaagattaaatctttaatgtacagggtgcaaaactcaagcgaatttgaactttacatctttgcaacaggaatgcacttaagtaaaaattttggctatacagttaaagaactttataaaaatggctttaaaaatatttatgaatttataaattatgataaatattatcaaactgataaggctttagctactacaattgatggattttcaaggtatgcaaatgagctaaaacctgatttaatcgtagtacatggagatagaattgagcctttagcagcagctattgttggagcattaaataatatcttagtagcgcatattgaaggcggagagatttcaggaactattgacgatagcttacgccacgctatatcaaaactagctcatattcatttagtaaatgatgagtttgcaaaaaggcgtttaatgcagcttggagaagatgaaaaatctatttttatcataggttcgcctgatttagaacttttaaacgataataaaatttcacttagcgaagcaaaaaaatattatgatataaattatgaaaactacgctttgcttatgtttcatcctgttacaactgaaattactagcattaaaaatcaagcagacaatttagtaaaagcactgatacaaagtaataaaaattatattgttatttatccaaataatgatttaggttttgaattaatcttgcaaagctatgaagagtttaaaaataaccctagatttaagctttttccatcgcttagatttgagtattttataactttgttaaaaaatgctgattttataataggtaattcaagttgtattttaaaagaggccttatacttaaaaacagcagggattttagttggctcaagacaaaatggaagacttggcaatgaaaatacactaaaagttaatgcaaatagtgatgaaatactaaaagctattaacactattcataaaaaacaagatttatttagcgctaagttagagattttagatagctcaaaattattttttgaatatttacaaagcggagatttttttaaactcagcacacaaaaagtttttaaggatataaaatgagcttagcaataatccctgctcgtggtggctcaaagggtattaaaaataaaaatttggttttattaaacaataaacctttaatttactacacgatcaaagctgcactaaatgctaaaagcattagtaaagttgttgtaagcagtgatagtgatgaaattttaaattatgcaaaaagtcaaaatgttgatattttaaaacgcccaattagccttgcacaagatgataccacaagcgataaagtgctgttacatgctctaaaattttataaagattatgaagatgtagtttttttacaacccacttcaccgctaagaacaaatattcatattaatgaagcttttaatctttataaaaatagcaatgcaaatgccctaattagcgtaagcgaatgtgataataaaattctaaaagcctttgtttgtaatgattgtggcgatttagcagggatttgtaatgatgaatatccttttatgccaaggcaaaaattgcctaaaacttatatgagcaatggtgcaatttatattttaaagataaaagaatttttaaacaatcctagctttttacaaagcaaaaccaagcattttttaatggacgaaagctcaagtttagatattgactgtttggaggatttaaaaaaggttgaacagatatggaaaaaataa

SEQ ID NO: 14 - protein, 394 aa, Rosenbergiella nectarea
MQSFTPPAPKGGNPVFMMFMLVTFFVSIAGALQAPTLSLYLSQELAAKPFMVGLFFTINAVTGIIISFILAKRSDRKGDRRRLLMFCCAMAIANALMFAFVRQYVVLITLGLILSALTSVVMPQLFALAREYADRTGREVVMFSSVMRTQMSLAWVIGPPISFALALNYGFITLYLVAAALFLLSLILIKTTLPSVPRLYPAEDLAKSAASGWKRTDVRFLFAASVLMWVCNLMYIIDMPLYISKSLGMPESFAGVLMGTAAGLEIPVMLLAGYLAKRVGKRPLVIVAAVCGLAFYPAMLVFHQQTGLLIIQLLNAVFIGIVAGLVMLWFQDLMPGKAGAATTLFTNSVSTGMIFAGLCQGLLSDLLGHQAIYVLATVLMVIALLLLLRVKEQA

SEQ ID NO: 15 - protein, 393 aa, Yersinia bercovieri
MKSALTFSRRINPVFLAFFVVAFLSGIAGALQAPTLSLFLSTEVKVRPLWVGLFYTVNAIAGITVSFILAKRSDSRGDRRKLIMVCYLMAVGNCLLFAFNRDYLTLITAGVLLASVANTAMPQIFALAREYADSSAREVVMFSSIMRAQLSLAWVIGPPLSFMLALNYGFTLMFSIAAGIFVLSALVVWFILPSVPRAEPVVDAPVVVQGSLFADKNVLLLFIASMLMWTCNTMYIIDMPLYITASLGLPERLAGLLMGTAAGLEIPIMLLAGYSVRYFGKRKIMLFAVLAGVLFYTGLVLFKFKTALMLLQIFNAIFIGIVAGIGMLYFQDLMPGRAGAATTLFTNSISTGVILAGVLQGGLTETWGHDSVYVMAMVLSILALIICARVREA

SEQ ID NO: 16 - protein, 393 aa, Yersinia frederiksenii
MKSALTFSRRINPVFLAFFVVAFLSGIAGALQAPTLSLFLSTEVKVRPLWVGLFYTVNAIAGITVSFVLAKRSDLRGDRRKLILVCYLMAVGNCLLFAFNRDYLTLITAGVLLAAVANTAMPQIFALAREYADNSAREVVMFSSIMRAQLSLAWVIGPPLSFMLALNYGFTLMFCIAAGIFVLSALVVWFILPSVQRAEPVMDAPTVAQGSLFADKDVLLLFIASMLMWTCNTMYIIDMPLYITASLGLPERLAGLLMGTAAGLEIPIMLLAGYSVRRFGKRKIMLFAVLAGVLFYTGLVLFKFKSALMLLQIFNAIFIGIVAGIGMLYFQDLMPGRAGAATTLFTNSISTGVILAGVLQGVLTETWGHNSVYVMAMILAILSLIICARVREA

SEQ ID NO: 17 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 18 - DNA, 107 bp, Escherichia coli
  source 1..107: mol_type = other DNA
tgtgagttagctcactcattaggcaccccaggctttacactttatgcttccggctcgtatgttgtgtggaattgtgagcggataacaatttcacacaggaaacagct

SEQ ID NO: 19 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagaaaaacagct

SEQ ID NO: 20 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactgagaaacagct

SEQ ID NO: 21 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaacaaaaaccggagatacc

SEQ ID NO: 22 - DNA, 141 bp, synthetic construct
  misc_feature 1..141: note = Promoter sequence
  source 1..141: mol_type = other DNA
tgtgagttagctcactcattaggcaccccaggctttacactttatgcttccggctcgtatgttgtgtggaatgcctacaagcatcgtggaggtccgtgactttcacgcatacaacaaacattaaccaaggaggaaacagct

SEQ ID NO: 23 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaaggaaaaacagct

SEQ ID NO: 24 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaactaggaaacagct

SEQ ID NO: 25 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaccgagaaacagct

SEQ ID NO: 26 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactaggaaacagct

SEQ ID NO: 27 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaagagcaaaacagct

SEQ ID NO: 28 - DNA, 203 bp, synthetic construct
  misc_feature 1..203: note = Promoter sequence
  source 1..203: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgtgcctacaagcatcgtggaggtccgtgactttcacgcatacaacaaacattaaccaaggaggaaacagct

SEQ ID NO: 29 - DNA, 189 bp, synthetic construct
  misc_feature 1..189: note = Promoter sequence
  source 1..189: mol_type = other DNA
gaaaacattcataaattaaatgtgaattgccgcacacattattaaataagatttacaaaatgttcaaaatgacgcatgaaatcacgtttcactttcgaattatgagcgaatatgcgcgatgcctacaagcatcgtggaggtccgtgactttcacgcatacaacaaacattaaccaaggaggaaacagct

SEQ ID NO: 30 - DNA, 239 bp, synthetic construct
  misc_feature 1..239: note = Promoter sequence
  source 1..239: mol_type = other DNA
ccatttagccatagtaaaaacatgaattgtttgatttcgcgcatattcgctcataattcgaaagtgaaacgtgatttcatgcgtcattttgaacattttgtaaatcttatttaataatgtgtgcggcaattcacatttaatttatgaatgttttcttaacatcgcggcatgcctacaagcatcgtggaggtccgtgactttcacgcatacaacaaacattaaccaaggaggaaacagct

SEQ ID NO: 31 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaactgagaaacagct

SEQ ID NO: 32 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaccgagaaacagct

SEQ ID NO: 33 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagaaaaacagct

SEQ ID NO: 34 - DNA, 350 bp, synthetic construct
  misc_feature 1..350: note = Promoter sequence
  source 1..350: mol_type = other DNA
tgcgtcgccattctgtcgcaacacgccagaatgcggcggcgatcactaactcaacaaatcaggcgatgtaaccgctttcaatctgtgagtgatttcacagtatcttaacaatgtgatagctatgattgcaccgttttaacgttgtaacccgtatgtaacagtgaataatcacttttgccgaggtaacagcgtcataacaacaattaaagccgttttctggagcgttaccgggcatggaagaacgaattttaaaaagtgagcttcggcgttcagtaacacttcattaactctactgccccgccgagcatttatctcaagcactaccctgcataagcaaggaggaaacagct

SEQ ID NO: 35 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaaggaaaaacagct

SEQ ID NO: 36 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagcaaaacagct

SEQ ID NO: 37 - DNA, 310 bp, synthetic construct
  misc_feature 1..310: note = Promoter sequence
  source 1..310: mol_type = other DNA
atgcgcaaatgcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagagctaaacagct

SEQ ID NO: 38 - DNA, 189 bp, synthetic construct; identical to SEQ ID NO: 29
  misc_feature 1..189: note = Promoter sequence
  source 1..189: mol_type = other DNA

SEQ ID NO: 39 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaagaacaaaacagct

SEQ ID NO: 40 - DNA, 300 bp, synthetic construct
  misc_feature 1..300: note = Promoter sequence
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaattcgaaacagct

(19) United States
(12) Patent Application Publication
(10) Pub. No.: US 2026/0028634 A1
(43) Pub. Date: Jan. 29, 2026

(54) NEW SIALYLTRANSFERASES FOR IN VIVO SYNTHESIS OF 3'SL AND 6'SL

(71) Applicant: DSM IP ASSETS B.V., HEERLEN (DK)

(72) Inventor: Manos PAPADAKIS, Hørsholm (DK)

(21) Appl. No.: 18/843,519

(22) PCT Filed: Mar. 1, 2023
(86) PCT No.: PCT/EP2023/055147

§ 371 Date: Sep. 3, 2024

(30) Foreign Application Priority Data
Mar. 2, 2022 (DK) PA202270076

(51) Int. Cl. C12N 15/70; C12N 9/10; C12P 19/18; C12R 1/19

(52) U.S. Cl. CPC C12N 15/70; C12N 9/1081; C12P 19/18; C12R 2001/19; C12Y 204/99001; C12Y 204/99004

(57) ABSTRACT

The present disclosure relates to the production of mixtures of sialylated Human Milk Oligosaccharides (HMOs), in particular 3′SL and 6′SL and genetically modified cells and their use in said production, where the cells express a heterologous sialyltransferase with dual α-2,3-sialyltransferase/α-2,6-sialyltransferase activity.

### FIELD

The present disclosure relates to the production of sialylated Human Milk Oligosaccharides (HMOs), in particular to the production of a mixture of 3′-sialyllactose (3′SL) and 6′-sialyllactose (6′SL) and to genetically engineered cells comprising a sialyltransferase with dual α-2,3-sialyltransferase/α-2,6-sialyltransferase activity for use in said production.

### BACKGROUND

The design and construction of bacterial cell factories to produce sialylated Human Milk Oligosaccharides (HMOs), especially to produce mixtures of sialylated Human Milk Oligosaccharides (HMOs), are of paramount importance in order to provide innovative and scalable solutions for the more complex products of tomorrow.

To this end, rational strain engineering principles are commonly applied to single bacterial cells. Such principles usually refer to a) the introduction of a desired biosynthetic pathway to the host, b) the increase of the cellular pools of relevant activated sugars required as donors in the desired reactions, c) the enhancement of lactose import by the native lactose permease LacY and d) the introduction of suitable glycosyltransferases to facilitate the biosynthetic production of sialylated oligosaccharides (for review see Bych et al 2019, Current Opinion in Biotechnology 56:130-137).

WO2007/101862 discloses production of sialylated HMOs using e.g., the sialyltransferase CstII with α-2,3 α-2,8-sialyltransferase activity from C. jejuni strain in combination with expression of the neuBCA genes to produce CMP-neu5AC. There is however no disclosure that CSTII produces a combination of 3′SL and 6′SL. Further attempts to produce sialylated HMOs are presented in WO2019/020707 and WO2019/118829, which describe sialyltransferases which are capable of producing both 3′SL and 6′SL, wherein 3′SL is a minor byproduct.

Production of mixtures of multiple sialylated HMOs further complicates the production, since it generally requires expression of multiple sialyltransferases which further challenges the growth and yield of the production strains.

In summary, in order to produce mixtures of sialylated HMOs, sialyltransferases that can sialylate more than one position on a given substrate would be advantageous and make it possible to obtain a scalable production platform capable of producing two or more sialylated HMOs.

### SUMMARY

The present disclosure relates to a genetically modified cell comprising a recombinant nucleic acid sequence encoding an enzyme with dual α-2,3-sialyltransferase and α-2,6-sialyltransferase activity, i.e., an α-2,3/6-sialyltransferase, capable of producing two or more sialylated HMOs, in particular 3′SL and 6′SL.

The present disclosure relates to the use of said genetically modified cell comprising sialyltransferases having dual activity, i.e., a sialyltransferase that acts on more than one position on a substrate, for the biosynthetic production of mixtures of sialylated HMOs. The ability of a group of sialyltransferases to act on a substrate in more than one way is a particularly powerful strain engineering tool since a cell modified to express a single heterologous sialyltransferase can produce more than one sialylated HMO product during a single fermentation process. In particular the α-2,3/6-sialyltransferases of the present disclosure are able to produce a mixture of 3′SL and 6′SL, wherein both are present in an amount above 20% of the total HMO produced. Furthermore, the metabolic burden entailed by the competition of enzymes for the same substrate is avoided.

A single-enzyme strain solution is highly advantageous compared to a multi-enzyme approach, enabling large scale production of mixtures of sialylated HMOs resulting in overall more stable growth and higher product yields.

In particular, the present disclosure relates to a genetically modified cell comprising a recombinant nucleic acid sequence encoding an enzyme with dual α-2,3-sialyltransferase/α-2,6-sialyltransferase activity, wherein said enzyme is selected from the group of sialyltransferases consisting of Chepa comprising or consisting of the amino acid sequence of SEQ ID NO: 1 or an amino acid sequence with at least 80%, such as at least 85%, such as at least 90%, such as at least 95% identity to SEQ ID NO: 1, Cinf1 comprising or consisting of the amino acid sequence of SEQ ID NO: 2 or an amino acid sequence with at least 80%, such as at least 85%, such as at least 90%, such as at least 95% identity to SEQ ID NO: 2, Ccol2 comprising or consisting of the amino acid sequence of SEQ ID NO: 3 or an amino acid sequence with at least 80%, such as at least 85%, such as at least 90%, such as at least 95% identity to SEQ ID NO: 3, Cjej1 comprising or consisting of the amino acid sequence of SEQ ID NO: 4 or an amino acid sequence with at least 80%, such as at least 85%, such as at least 90%, such as at least 95% identity to SEQ ID NO: 4, Poral2 comprising or consisting of the amino acid sequence of SEQ ID NO: 5 or an amino acid sequence with at least 80%, such as at least 85%, such as at least 90%, such as at least 95% identity to SEQ ID NO: 5 and CstII comprising or consisting of the amino acid sequence of SEQ ID NO: 6 or an amino acid sequence with at least 80%, such as at least 85%, such as at least 90%, such as at least 95% identity to SEQ ID NO: 6, and wherein said cell produces 3′SL and 6′SL.

The genetically modified cell according to the present disclosure can further comprise a promoter element that controls the expression of the recombinant nucleic acid encoding an enzyme with α-2,3-sialyltransferase and α-2,6-sialyltransferase activity. The sialyltransferase may e.g., be under the control of a promoter selected from the group consisting of PglpF, PglpA_70UTR, PglpT_70UTR, Plac, PmglB_70UTR and variants thereof with a nucleic acid sequence selected from the group consisting of SEQ ID NOs 17 to 40. Preferably, the sialyltransferase is under the control of a recombinant promoter selected from the group consisting of SEQ ID NOs 17, 19, 20, 21, 22, 23, 24, 24, 26, 27, 28, 29, 30, 31, 32, 33, 34, 35, and 36, respectively.

The genetically modified cell according to the present disclosure can further comprise a nucleic acid sequence encoding an MFS transporter protein capable of exporting the sialylated HMO into the extracellular medium. Preferably, the MFS transporter protein is the Nec or YberC or Fred protein, with an amino acid sequence according to SEQ ID NOs: 14 or 15 or 16, respectively.

The genetically modified cell according to the present disclosure can comprise a biosynthetic pathway for making a sialic acid sugar nucleotide, such as CMP-Neu5Ac. Said sialic acid sugar nucleotide pathway can be encoded by the nucleic acid sequence encoding NeuBCA from

Campylobacter jejuni (SEQ ID NO: 13). The nucleic acid sequence encoding NeuBCA, can be encoded from a high-copy plasmid bearing the neuBCA operon.

The genetically modified cell according to the present disclosure can be a microorganism, such as a bacterium or a fungus, wherein said fungus can be selected from a yeast cell, such as of the genera Komagataella, Kluyveromyces, Yarrowia, Pichia, Saccaromyces, Schizosaccharomyces or Hansenula, or from a filamentous fungous of the genera Aspargillus, Fusarium or Thricoderma, and said bacterium can be selected from the exemplified group consisting of Escherichia sp., Bacillus sp., Corynebacterium sp., Lactobacillus sp. and Campylobacter sp. Accordingly, the genetically modified cell according to the present disclosure can be E coli.

The genetically modified cell of the present disclosure can be used in the production of a mixture of sialylated HMO, in particular for the production of a mixture of 3′SL and 6′SL.

Accordingly, the present disclosure also relates to a method for producing at least two different sialylated human milk oligosaccharides (HMOs), said method comprising culturing a genetically modified cell comprising a recombinant nucleic acid sequence encoding an enzyme with dual α-2,3-sialyltransferase/α-2,6-sialyltransferase activity, wherein said enzyme is selected from the group consisting of Chepa, Cinf1, Ccol2, Cjej1, Poral2 and CstII, wherein the sialylated human milk oligosaccharides (HMOs) produced are 3′SL and 6′SL.

In addition, the disclosure also relates to a recombinant nucleic acid sequence encoding an enzyme with dual α-2,3-sialyltransferase/α-2,6-sialyltransferase activity, wherein said recombinant nucleic acid sequence is selected from the group consisting of Chepa comprising or consisting of the amino acid sequence of SEQ ID NO: 1 or an amino acid sequence with at least 80%, such as at least 85%, such as at least 90%, such as at least 95% identity to SEQ ID NO: 1, Cinf1 comprising or consisting of the amino acid sequence of SEQ ID NO: 2 or an amino acid sequence with at least 80%, such as at least 85%, such as at least 90%, such as at least 95% identity to SEQ ID NO: 2, Ccol2 comprising or consisting of the amino acid sequence of SEQ ID NO: 3 or an amino acid sequence with at least 80%, such as at least 85%, such as at least 90%, such as at least 95% identity to SEQ ID NO: 3, Cjej1 comprising or consisting of the amino acid sequence of SEQ ID NO: 4 or an amino acid sequence with at least 80%, such as at least 85%, such as at least 90%, such as at least 95% identity to SEQ ID NO: 4, Poral2 comprising or consisting of the amino acid sequence of SEQ ID NO: 5 or an amino acid sequence with at least 80%, such as at least 85%, such as at least 90%, such as at least 95% identity to SEQ ID NO: 5, and CstII comprising or consisting of the amino acid sequence of SEQ ID NO: 16 or an amino acid sequence with at least 80%, such as at least 85%, such as at least 90%, such as at least 95% identity to SEQ ID NO: 6, wherein the enzyme encoding sequence is preferably under the control of a promoter sequence, such as a promoter selected from the group consisting of PglpF, Plac, PmglB_70UTR, PglpA_70UTR, PglpT_70UTR (SEQ ID NOs: 17, 18, 28, 29, 30, respectively and variants thereof as disclosed in table 2 (SEQ ID NOs: 19-27 and 31-40). Preferably, the sialyltransferase is under the control of a strong recombinant promoter selected from the group consisting of SEQ ID NOs 17, 19, 20, 21, 22, 23, 24, 24, 26, 27, 28, 29, 30, 31, 32, 33, 34, 35 and 36. In particular the nucleic acid sequence encoding CstII is under the control of a promoter selected from the group consisting of PglpF, PmglB_70UTR PglpA_70UTR and PglpT_70UTR with a nucleic acid sequence according to SEQ ID Nos: 17, 28, 29, 30, respectively, or variants thereof disclosed in table 2. Said nucleic acid construct is typically used in a host cell for producing at least two different sialylated HMOs, such as 3′SL and 6′SL. Various exemplary embodiments and details are described hereinafter, with reference to the figures and sequences when relevant. It should be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### BRIEF DESCRIPTION OF THE FIGURES

FIG. 1

Genetically modified cells expressing an enzyme with a dual α-2,3-sialyltransferase and α-2,6-sialyltransferase activity that produce both 3′SL and 6′SL, in a different ratio depending on the enzyme.

### DETAILED DESCRIPTION

The present disclosure approaches the biotechnological challenges of in vivo HMO production, in particular of sialylated HMOs that contain a sialyl monosaccharide, such as the sialylated HMOs 3′SL and 6′SL. The present disclosure offers specific strain engineering solutions to produce specific sialylated HMOs, in particular a mixture of 3′SL and 6′SL, by exploiting the dual activity of the α-2,3/6-sialyltransferases of the present disclosure, which provides a simpler, production of sialylated HMO mixtures relying on a single sialyltransferase with dual activity, which reduces the metabolic burden of expression of multiple glycosyltransferases.

In a currently preferred embodiment, in addition to the gene for the dual-2,3/6-sialyltransferase, the genetically modified cell covered by the present disclosure expresses genes encoding a biosynthetic pathway for making a sialic acid sugar nucleotide, such as the neuBCA operon from Campylobacter jejuni shown in SEQ ID NO: 13, which enables the cell to produce a sialylated oligosaccharide from substrates, such as lactose and nucleotide-activated sugars, such as in particular CMP-N-acetylneuraminic acid.

In particular, the sialylated HMO(s) produced are a mixture of 3′SL and 6′SL.

The advantage of using any one of the α-2,3/6-sialyltransferases of the present disclosure in the present context is their ability to recognize, and sialylate, lactose in two positions, in particular at position C6 or C3 of the galactose unit in lactose. I.e., in particular to generate 3′SL and 6′SL, thus producing a mixture of sialylated HMOs. To our knowledge, the α-2,3/6-sialyltransferases of the present disclosure do not add two sialyl moieties to the same galactose unit. Any one of the enzymes presented herein allow for production of a mixture of HMOs comprising 3′SL and 6′SL, preferably with 3′SI and 6′SL being the only HMOs in the mixture, and preferably wherein at least 20% of the total molar HMO content produced by the cell is 3′SL and 20% of the total molar HMO content produced by the cell is 6′SL. In particular, the present disclosure describes α-2,3/6-sialyltransferases that produce mixtures of 3′SL and 6′SL, with different ratios of the two sialylated HMOs, depending on the specific enzyme. This allows for a tailored production of specific mixtures of HMOs, wherein the molar ratio of the sialylated HMOs can be optimized in the production step. The traits of the α-2,3/6-sialyltransferases described herein are therefore well-suited for large-scale industrial production of mixtures of 3′SL and 6′SL.

The genetically modified cells of the present disclosure, which express a selective α-2,3/6-sialyltransferase with high specificity for both 3′ and 6′ sialylation of lactose, for the first time enable the production of mixtures of 3′SL and 6′SL following expression of a single enzyme.

Thereby, the present disclosure enables a more efficient biotechnological production of mixtures of 3′SL and 6′SL.

In the following sections, individual elements of the disclosure and in, particular of the genetically modified cell is described, it is understood that these elements can be combined across the individual sections.

### Oligosaccharides

In the present context, the term “oligosaccharide” means a sugar polymer containing at least three monosaccharide units, i.e., a tri-, tetra-, penta-, hexa-or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. N-acetylneuraminic acid). Preferably, the oligosaccharide is an HMO, in particular an HMO composed of three monosaccharide units.

### Human Milk Oligosaccharide (HMO)

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl unit, and this core structure can be substituted by an α-L-fucopyranosyl and/or an α-N-acetyl-neuraminyl (sialyl) moiety. HMO structures are e.g., disclosed by Xi Chen in Chapter 4 of Advances in Carbohydrate Chemistry and Biochemistry 2015 vol 72.

The present disclosure focuses on sialylated HMO's, which are generally acidic. Examples of acidic HMOs include 3′-sialyllactose (3′SL), 6′-sialyllactose (6′SL), 3-fucosyl-3′-sialyllactose (FSL), 3′-O-sialyllacto-N-tetraose a (LST-a), fucosyl-LST-a (FLST-a), 6′-O-sialyllacto-N-tetraose b (LST-b), fucosyl-LST b (FLST b), 6′-O-sialyllacto-N-neotetraose (LST-c), fucosyl-LST-c (FLST-c), 3′-O-sialyllacto-N-neotetraose (LST-d), fucosyl-LST d (FLST-d), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

In one aspect of the present disclosure, the sialylated human milk oligosaccharide (HMO) produced by the cell is a sialylated HMO selected from the group consisting of 3′SL and 6′SL. In a further aspect of the present disclosure, the sialylated human milk oligosaccharide (HMO) produced by the cell is a mixture of two HMOs, each of three monosaccharide units, such as 3′SL and 6′SL.

Production of some of the above mentioned sialylated HMO's may require the presence of two or more glycosyltransferase activities, in particular if starting from lactose as the acceptor oligosaccharide or if preparing a mixture of HMOs. This is however not the case for the production for 3′SL and 6′SL, which in the present disclosure only requires expression of a single glycosyltransferase with dual α-2,3-sialyltransferase and α-2,6-sialyltransferase activity.

### An acceptor oligosaccharide

A genetically modified cell according to the present disclosure comprises a recombinant nucleic acid sequence encoding an enzyme with dual α-2,3-sialyltransferase and α-2,6-sialyltransferase activity capable of transferring sialic acid from an activated sugar to the terminal galactose of an acceptor oligosaccharide.

In the context of the present disclosure, an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making an HMO and can also be termed the precursor molecule.

The acceptor oligosaccharide can be either an intermediate product of the present fermentation process, an end-product of a separate fermentation process employing a separate genetically modified cell, or an enzymatically or chemically produced molecule.

In the present context, said acceptor oligosaccharide is preferably lactose for the production of 3′SL and 6′SL, such as for mixtures of 3′SL and 6′SL. The precursor molecule is preferably fed to the genetically modified cell which is capable of producing the sialylated HMO from the precursor.

### Glycosyltransferases

The genetically modified cell according to the present disclosure comprises at least one recombinant nucleic acid sequence encoding at least one glycosyltransferase capable of transferring a sialyl residue from a sialyl donor to an acceptor oligosaccharide to synthesize a sialylated human milk oligosaccharide product, i.e., a sialyltransferase.

In the present context, the genetically modified cell according to the present disclosure comprises at least one recombinant nucleic acid sequence encoding at least one sialyltransferase with dual α-2,3-sialyltransferase and α-2,6-sialyltransferase activity, i.e., a α-2,3/6-sialyltransferase.

The genetically modified cell, according to the present disclosure optimally comprise at least one nucleic acid sequence encoding the sialyltransferase with dual activity, which is capable of transferring a sialyl residue from a sialyl donor to an acceptor oligosaccharide, preferably to lactose.

The sialyltransferase in the genetically modified cell of the present disclosure is an α-2,3/6-sialyltransferase. Preferably, the α-2,3/6-sialyltransferase is capable of transferring a sialic acid unit onto the terminal galactose of a lactose molecule. Specifically, the α-2,3/6-sialyltransferase is capable of transferring a sialic acid unit onto the terminal galactose of a lactose molecule in the 3′ position or in the 6′ position.

Typically, the genetically modified cell of the present disclosure produces a mixture of sialylated human milk oligosaccharides (HMOs) wherein the molar content of each HMO in the mixture is above 20% of the total molar content of HMO produced by the cell.

In one aspect, the genetically modified cell of the disclosure comprises a recombinant nucleic acid sequence encoding an enzyme with dual α-2,3-sialyltransferase/α-2,6-sialyltransferase activity, wherein said cell is capable of producing at least 20% 3′SL and 20% 6′SL of the total molar HMO content produced by the cell.

In one embodiment, the genetically modified cell of the present disclosure, expressing an α-2,3/6-sialyltransferase produces 3′SL and 6′SL, wherein the molar ratio of the produced sialylated human milk oligosaccharides (HMOs) 3′SL: 6′SL is between 20:80 and 80:20, such as between 25:75 and 75:25, such as 25:75, 30:70, 35:65, 40:60, 45:55, 50:50, 55:45, 60:40,65:35, 70:30 or such as 75:25.

A genetically modified cell, according to the present disclosure may comprise additional glycosyltransferases, for the production of complex mixtures comprising additional HMOs besides 3′SL and 6′SL. The additional glycosyltransferase is preferably selected from the group consisting of, fucosyltransferases, galactosyltransferases, glucosaminyltransferases, sialyltransferases, N-acetylglucosaminyl transferases and N-acetylglucosaminyl transferases.

In one embodiment, the expression of an α-2,3/6-sialyltransferase of the present disclosure is further combined with a β-1,3-galactosyltransferase, such as galTK from Helicobacter pylori (GenBank accession nr WP_111735921). In a further embodiment, a third enzyme is expressed, such as α-1,3-N-acetyl-glucosaminyl-transferase, e.g., LgtA from Neisseria meningitidis (GenBank accession nr WP_002248149.1). In such a genetically modified cell, the mixture of HMOs produced will comprise the sialylated HMOs 3′SL and LSTa, this is particularly true when the cell comprises the α-2,3/6-sialyltransferase Ccol2, Chepa and Cjej1.

Exemplified glycosyltransferases are preferably selected from the glycosyltransferases described below. α-2,3/6-sialyltransferase

An α-2,3/6-sialyltransferase refers to a glycosyltransferase that catalyzes the transfer of sialyl from a donor substrate, such as CMP-N-acetylneuraminic acid, to an acceptor molecule in an α-2,3-linkage or an α-2,6-linkage, and which is capable of both. Preferably, an α-2,3/6-sialyltransferase used herein does not originate in the species of the genetically engineered cell, i.e., the gene encoding the α-2,3/6-sialyltransferase is of heterologous origin and is selected from an α-2,3/6-sialyltransferase identified in table 1.

In the present disclosure, the α-2,3/6-sialyltransferase expressed in the genetically modified cell is selected from the group consisting of Chepa, Cinf1, Ccol2, Cjej1, Poral2 and CstII (table 1). Expression of any one or a combination of these enzymes in a genetically modified cell of the present disclosure is used to produce a mixture of sialylated HMOs, such as a mixture of 3′SL and 6′SL.

TABLE 1
List of α-2,3/6-sialyltransferase enzymes capable of producing 3′SL and 6′SL.

| Enzyme | | SEQ ID | | |
|---|---|---|---|---|
| Name | GenBank ID | NO: | Origin | Ref |
| Chepa | WP_066776435.1 | 1 | Campylobacter hepaticus | — |
| Cinf1 | WP_011272254.1 | 2 | Haemophilus influenzae | — |
| Ccol2 | EAH6554614.1 | 3 | Campylobacter coli | — |
| Cjej1 | EBD1936710.1 | 4 | Campylobacter jejuni | — |
| Poral2 | WP_101774701.1 | 5 | Pasteurella oralis | — |
| Cstll | AAF31771.1 | 6 | Campylobacter jejuni | WO2007/101862 |
| | | | | WO2019/020707 |

The GenBank IDs reflect the full-length enzymes. In the present disclosure truncated or mutated versions may have been used, these are represented by the SEQ ID NOs. The α-2,3/6-sialyltransferase CstII is known from the prior art as a α-2,3 α-2,8-sialyltransferase, it has however not been disclosed to produce a mixture of 3′SL and 6′SL.

Example 1 of the present disclosure has identified the heterologous α-2,3/6-sialyltransferases Chepa, Cinf1, Ccol2, Cjej1, Poral2 and CstII (SEQ ID NO: 1, 2, 3, 4, 5 and 6 respectively), as α-2,3/6-sialyltransferases which are capable of producing mixtures of 3′SL and 6′SL in different ratios when introduced into a genetically modified cell.

In one embodiment of the disclosure, the enzyme with dual α-2,3-sialyltransferase and α-2,6-sialyltransferase activity is Chepa from Campylobacter hepaticus, comprising or consisting of the amino acid sequence of SEQ ID NO: 1, or an amino acid sequence with at least 80%, such as at least 85%, such as at least 90%, such as at least 95%, or such as at least 99% identity to SEQ ID NO: 1.

In another embodiment of the disclosure, the enzyme with dual α-2,3-sialyltransferase and α-2,6-sialyltransferase activity is Cinf1 from Haemophilus influenzae, comprising or consisting of the amino acid sequence of SEQ ID NO: 2, or an amino acid sequence with at least 80%, such as at least 85%, such as at least 90%, such as at least 95%, or such as at least 99% identity to SEQ ID NO: 2.

In another embodiment of the disclosure, the enzyme with dual α-2,3-sialyltransferase and α-2,6-sialyltransferase activity is Ccol2 from Campylobacter coli, comprising or consisting of the amino acid sequence of SEQ ID NO: 3, or an amino acid sequence with at least 80%, such as at least 85%, such as at least 90%, such as at least 95%, or such as at least 99% identity to SEQ ID NO: 3.

In another embodiment of the disclosure, the enzyme with dual α-2,3-sialyltransferase and α-2,6-sialyltransferase activity is Cjej1 from Campylobacter jejuni, comprising or consisting of the amino acid sequence of SEQ ID NO: 4, or an amino acid sequence with at least 80%, such as at least 85%, such as at least 90%, such as at least 95%, or such as at least 99% identity to SEQ ID NO: 4.

In another embodiment of the disclosure, the enzyme with dual-2,3-sialyltransferase and α-2,6-sialyltransferase activity is Poral2 from Pasteurella oralis, comprising or consisting of the amino acid sequence of SEQ ID NO: 5, or an amino acid sequence with at least 80%, such as at least 85%, such as at least 90%, such as at least 95%, or such as at least 99% identity to SEQ ID NO: 5.

In another embodiment of the disclosure, the enzyme with dual α-2,3-sialyltransferase and α-2,6-sialyltransferase activity is CstII from Campylobacter jejuni, comprising or consisting of the amino acid sequence of SEQ ID NO: 6, or an amino acid sequence with at least 80%, such as at least 85%, such as at least 90%, such as at least 95%, or such as at least 99% identity to SEQ ID NO: 6.

In one embodiment the genetically engineered cell comprises a α-2,3/6-sialyltransferase selected from Chepa or Cinf1 or Ccol2 as defined above, wherein the cell produces 3′SL and 6′SL, and the 3′SL: 6′SL ratio is between 80:55 and 75:60, specifically there is always at least 5%, such as 10% or 20% more 3′SL than 6′SL.

In another embodiment the genetically engineered cell comprises a α-2,3/6-sialyltransferase selected from Poral2 or Cjej1 as defined above, wherein the cell produces 3′SL and 6′SL, and the 3′SL: 6′SL ratio is between 20:80 and 30:70, specifically there is always at least 5%, such as 10% or 20% more 6′SL than 3′SL.

In one embodiment, the genetically modified cell of the present disclosure comprises the sialyltransferase Chepa comprising or consisting of the amino acid sequence of SEQ ID NO: 1, or an amino acid sequence with at least 80%, such as at least 85%, such as at least 90%, such as at least 95% identity to SEQ ID NO: 1, and produces 3′SL and 6′SL, wherein the molar ratio of the produced 3′SL and 6′SL is between 80:20 and 70:30, such as approximately 75:25.

In one embodiment, the genetically modified cell of the present disclosure comprises the sialyltransferase Cinf1 comprising or consisting of the amino acid sequence of SEQ ID NO: 2 or an amino acid sequence with at least 80%, such as at least 85%, such as at least 90%, such as at least 95% identity to SEQ ID NO: 2, and produces 3′SL and 6′SL, wherein the molar ratio of the produced 3′SL and 6′SL is between 70:30 and 60:40, such as approximately 65:35.

In one embodiment, the genetically modified cell of the present disclosure comprises the sialyltransferase Ccol2 comprising or consisting of the amino acid sequence of SEQ ID NO: 3 or an amino acid sequence with at least 80%, such as at least 85%, such as at least 90%, such as at least 95% identity to SEQ ID NO: 3, and produces 3′SL and 6′SL, wherein the molar ratio of the produced 3′SL and 6′SL is between 65:35 and 55:45, such as approximately 60:40.

In one embodiment, the genetically modified cell of the present disclosure comprises the sialyltransferase Cjej1 comprising or consisting of the amino acid sequence of SEQ ID NO: 4 or an amino acid sequence with at least 80%, such as at least 85%, such as at least 90%, such as at least 95% identity to SEQ ID NO: 4, and produces 3′SL and 6′SL, wherein the molar ratio of the produced 3′SL and 6′SL is between 30:70 and 40:60, such as approximately 35:65.

In one embodiment, the genetically modified cell of the present disclosure comprises the sialyltransferase Poral2 comprising or consisting of the amino acid sequence of SEQ ID NO: 5 or an amino acid sequence with at least 80%, such as at least 85%, such as at least 90%, such as at least 95% identity to SEQ ID NO: 5, and produces 3′SL and 6′SL, wherein the molar ratio of the produced 3′SL and 6′SL is between 25:75 and 40:60, such as approximately 35:65.

In one embodiment, the genetically modified cell of the present disclosure comprises the sialyltransferase CstII comprising or consisting of the amino acid sequence of SEQ ID NO: 6 or an amino acid sequence with at least 80%, such as at least 85%, such as at least 90%, such as at least 95% identity to SEQ ID NO: 6, and produces 3′SL and 6′SL, wherein the molar ratio of the produced 3′SL and 6′SL is between 40:60 and 60:40, such as approximately 50:50.

### Glycosyl-Donor-Nucleotide-Activated Sugar Pathways

When carrying out the method of the present disclosure, preferably a glycosyltransferase mediated glycosylation reaction takes place in which an activated sugar nucleotide serves as glycosyl-donor. An activated sugar nucleotide generally has a phosphorylated glycosyl residue attached to a nucleoside. A specific glycosyl transferase enzyme accepts only a specific sugar nucleotide. Thus, preferably, the following activated sugar nucleotides are involved in the glycosyl transfer: glucose-UDP-GIcNAc, UDP-galactose, UDP-glucose, UDP-N-acetylglucosamine, UDP-N-acetylgalactosamine (GIcNAc) and CMP-N-acetylneuraminic acid. The genetically modified cell according to the present disclosure can comprise one or more pathways to produce a nucleotide-activated sugar selected from the group consisting of glucose-UDP-GIcNAc, GDP-fucose, UDP-galactose, UDP-glucose, UDP-N-acetylglucosamine, UDP-N-acetylgalactosamine and CMP-N-acetylneuraminic acid.

In one embodiment of the method, the genetically modified cell is capable of producing one or more activated sugar nucleotides mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, sucrose, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).

The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharides for sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the host cell.

### Sialic Acid Sugar Nucleotide Synthesis Pathway

Preferably, the genetically modified cell according to the present disclosure comprises a sialic acid sugar nucleotide synthesis capability, i.e., the genetically modified cell comprises a biosynthetic pathway for making a sialate sugar nucleotide, such as CMP-N-acetylneuraminic acid as glycosyl-donor for the α-2,3/6-sialyltransferase of the present disclosure. E.g., the genetically modified cell comprises a sialic acid synthetic capability through provision of an exogenous UDP-GlcNAc 2-epimerase (e.g., NeuC of Campylobacter jejuni (GenBank AAK91727.1) or equivalent (e.g., (GenBank CAR04561.1), a Neu5Ac synthase (e.g., NeuB of C. jejuni (GenBank AAK91726.1) or equivalent, (e.g., Flavobacterium limnosediminis sialic acid synthase, GenBank WP_023580510.1), and/or a CMP-Neu5Ac synthetase (e.g., NeuA of C. jejuni (GenBank AAK91728.1) or equivalent, (e.g., Vibrio brasiliensis CMP-sialic acid synthase, GenBank WP_006881452.1).

In one or more examples UDP-GlcNAc 2-epimerase, CMP-Neu5Ac synthetase, Neu5Ac synthase from Campylobacter jejuni, also referred to as NeuBCA from Campylobacter jejuni or simply the neuBCA operon, may be plasmid borne or integrated into the genome of the genetically modified cell. Preferably, the sialic acid sugar nucleotide pathway is encoded by the nucleic acid sequence encoding NeuBCA from Campylobacter jejuni (SEQ ID NO: 13) or a functional variant thereof having an amino acid sequence which is at least 80% identical, such as at least 85%, such as at least 90% or such as at least 99% to SEQ ID NO: 13.

Additionally, the nucleic acid sequence encoding NeuBCA is preferably encoded from a high-copy plasmid bearing the neuBCA operon. In embodiments, the high-copy plasmid is the BlueScribe M13 plasmid (pBS). In relation to the present disclosure, a high-copy plasmid is a plasmid that that replicates to a copy number above 50 when introduced into the cell.

### A deficient sialic acid catabolic pathway

The genetically modified cell of the present disclosure preferably has a deficient sialic acid catabolic pathway. By “sialic acid catabolic pathway” is meant a sequence of reactions, usually controlled, and catalysed by enzymes, which results in the degradation of sialic acid. An exemplary sialic acid catabolic pathway described hereafter is the E. coli pathway. In this pathway, sialic acid (Neu5Ac; N-acetylneuraminic acid) is degraded by the enzymes NanA (N-acetylneuraminic acid lyase) and NanK (N-acetylmannosamine kinase) and NanE (N-acetylmannosamine-6-phosphate epimerase), all encoded from the nanATEK-yhcH operon, and repressed by NanR (http://ecocyc.org/ECOLI). A deficient sialic acid catabolic pathway is rendered in the E. coli host by introducing a mutation in the endogenous nanA (N-acetylneuraminate lyase) (e.g., GenBank Accession Number D00067.1 (GL216588)) and/or nanK (N-acetylmannosamine kinase) genes (e.g., GenBank Accession Number (amino acid) BAE77265.1 (GL85676015)), and/or nanE (N-acetylmannosamine-6-phosphate epimerase, GI: 947745), incorporated herein by reference). Optionally, the nanT (N-acetylneuraminate transporter) gene is also inactivated or mutated. Other intermediates of sialic acid metabolism include: (ManNAc-6-P) N-acetylmannosamine-6-phosphate; (GlcNAc-6-P) N-acetylglucosamine-6-phosphate; (GlcN-6-P) Glucosamine-6-phosphate, and (Fruc-6-P) Fructose-6-phosphate. In some preferred embodiments, nanA is mutated. In other preferred embodiments, nanA and nanK are mutated, while nanE remains functional. In another preferred embodiment, nanA and nanE are mutated, while nanK has not been mutated, inactivated or deleted. A mutation is one or more changes in the nucleic acid sequence coding the gene product of nanA, nank, nanE, and/or nanT. E.g., the mutation may be 1, 2, up to 5, up to 10, up to 25, up to 50 or up to 100 changes in the nucleic acid sequence. E.g., the nanA, nank, nanE, and/or nanT genes are mutated by a null mutation. Null mutations as described herein encompass amino acid substitutions, additions, deletions, or insertions, which either cause a loss of function of the enzyme (i.e., reduced or no activity) or loss of the enzyme (i.e., no gene product). By “deleted” is meant that the coding region is removed completely or in part such that no (functional) gene product is produced. By inactivated is meant that the coding sequence has been altered such that the resulting gene product is functionally inactive or encodes for a gene product with less than 100%, e.g., 90%, 80%, 70%, 60%, 50%, 40%, 30% or 20% of the activity of the native, naturally occurring, endogenous gene product. Thus, in the present disclosure, nanA, nank, nanE, and/or nanT genes are preferably inactivated.

### Major facilitator superfamily (MFS) transporter proteins

The oligosaccharide product, the HMO produced by the cell, can be accumulated both in the intra-and the extracellular matrix. The product can be transported to the supernatant in a passive way, i.e., it diffuses outside across the cell membrane. Alternatively, the HMO transport can be facilitated by major facilitator superfamily transporter proteins that promote the effluence of sugar derivatives from the cell to the supernatant. The major facilitator superfamily transporter can be present exogenously or endogenously and is overexpressed under the conditions of the fermentation to enhance the export of the oligosaccharide derivative (HMO) produced. The specificity towards the sugar moiety of the product to be secreted can be altered by mutation by means of known recombinant DNA techniques.

Thus, the genetically modified cell according to the present disclosure can further comprise a nucleic acid sequence encoding a major facilitator superfamily transporter protein capable of exporting the sialylated human milk oligosaccharide product or products.

In the recent years, several new and efficient major facilitator superfamily transporter proteins have been identified, each having specificity for different recombinantly produced HMOs and development of recombinant cells expressing said proteins are advantageous for high scale industrial HMO manufacturing. E.g., WO2021/123113 discloses different E. coli and heterologous transporters for the export of 3′SL and 6′SL

Thus, in one or more exemplary embodiments, the genetically engineered cell according to the method described herein further comprises a gene product that acts as a major facilitator superfamily transporter. The gene product that acts as a major facilitator superfamily transporter may be encoded by a recombinant nucleic acid sequence that is expressed in the genetically engineered cell. The recombinant nucleic acid sequence encoding a major facilitator superfamily transporter, may be integrated into the genome of the genetically engineered cell, or expressed using a plasmid.

In one embodiment, the genetically modified cell of the disclosure comprises a nucleic acid sequence encoding a major facilitator superfamily transporter protein capable of exporting the sialylated human milk oligosaccharide product into the extracellular medium, in particular, a transporter with specificity towards 3′SL and/or 6′SL is preferred.

### Nec

In an embodiment, the genetically modified cell of the disclosure comprises a nucleic acid sequence encoding an efflux transporter protein capable of exporting the sialylated human milk oligosaccharide product, such as 3′SL and 6′SL, into the extracellular medium. In the current context, said efflux transporter protein is preferably a heterologous gene encoding a putative MFS (major facilitator superfamily) transporter protein, originating from the bacterium Rosenbergiella nectarea. More specifically, the disclosure relates to a genetically modified cell optimized to produce an oligosaccharide, in particular a sialylated HMO, comprising a recombinant nucleic acid encoding a protein having at least 80%, such as at least 85%, such as at least 90% such as at least 95% or 100% sequence identity to the amino acid sequence of the amino acid sequence having GenBank accession ID WP_092672081.1 or SEQ ID NO: 14.

Additionally, the MFS transporter protein with the GenBank accession ID WP_092672081.1 is further described in WO2021/148615 and is identified herein as “Nec protein” or “Nec transporter” or “Nec”, interchangeably; a nucleic acid sequence encoding Nec protein is identified herein as “nec coding nucleic acid/DNA” or “nec gene” or “nec”.

Nec is expected to facilitate an increase in the efflux of the produced sialylated HMOs, e.g., 3′SL and 6′SL in the genetically engineered cells of the current disclosure.

Accordingly, in an embodiment, the genetically modified cell of the present disclosure comprises a nucleic acid sequence encoding the Nec transporter protein.

### Fred/YberC

In embodiments, the genetically modified cell of the present disclosure comprises a nucleic acid sequence encoding an efflux transporter protein capable of exporting the simple sialylated human milk oligosaccharide product such as 3′SL and 6′SL into the extracellular medium. In the current context, said efflux transporter protein is preferably a heterologous gene encoding a putative MFS (major facilitator superfamily) transporter protein, originating from the bacterium Yersinia frederiksenii and/or the bacterium Yersinia bercovieri. More specifically, the disclosure relates to a genetically modified cell optimized to produce an oligosaccharide, in particular a sialylated HMO, comprising a recombinant nucleic acid encoding a protein having at least 80%, such as at least 85%, such as at least 90% such as at least 95% or 100% sequence identity to the amino acid sequence of the amino acid sequence having the GenBank accession ID WP_087817556.1 (or SEQ ID NO: 16) or GenBank accession EEQ08298 (or SEQ ID NO: 15).

The MFS transporter protein with the GenBank accession ID WP_087817556.1 is further described in WO2021/148620 and is identified herein as “Fred protein” or “Fred transporter” or “Fred”, interchangeably; a nucleic acid sequence encoding Fred protein is identified herein as “fred coding nucleic acid/DNA” or “fred gene” or “fred”.

Accordingly, in an embodiment, the genetically modified cell of the present disclosure comprises a nucleic acid sequence encoding the Nec or Fred transporter protein.

Additionally, the MFS transporter protein with the GenBank accession ID EEQ08298 is further described in WO2021148610 and is identified herein as “YberC protein” or “YberC transporter” or “YberC”, interchangeably; a nucleic acid sequence encoding YberC protein is identified herein as “YberC coding nucleic acid/DNA” or “yberC gene” or “yberC”.

Fred and YberC facilitate an increase in the efflux of the produced sialylated HMOs, e.g., 3′SL and 6′SL in the genetically engineered cells of the current disclosure.

Accordingly, in an embodiment, the genetically modified cell of the present disclosure comprises a nucleic acid sequence encoding the Fred transporter protein. In an embodiment, the genetically modified cell of the present disclosure comprises a nucleic acid sequence encoding the YberC transporter protein.

### The genetically modified cell

In the present context, the terms “a genetically modified cell” and “a genetically engineered cell” are used interchangeably. As used herein “a genetically modified cell” is a host cell whose genetic material has been altered by human intervention using a genetic engineering technique, such a technique is e.g., but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetic modifications can e.g., be selected from inclusion of glycosyltransferases, and/or metabolic pathway engineering and inclusion of MFS transporters as described in the above sections, which the skilled person will know how to combine into a genetically modified cell capable of producing one or more sialylated HMO's.

In one aspect of the disclosure, the genetically modified cell comprises a recombinant nucleic acid sequence encoding an enzyme with α-2,3-sialyltransferase and α-2,6-sialyltransferase activity, which is capable of producing a mixture of 3′SL and 6′SL, in particular, the genetically modified cell is capable of producing at least 20% 3′SL and 20% 6′SL of the total molar HMO content produced by the cell. More specifically, the molar ratio of the produced 3′SL and 6′SL is between 25:75 and 80:20, such as 25:75, 30:70, 35:65, 40:60, 45:55, 50:50, 55:45, 60:40, 65:35, 70:30, 75:25 or such as 80:20.

The genetically engineered cell is preferably a microbial cell, such as a prokaryotic cell or eukaryotic cell.

The genetically engineered cell may be e.g., a bacterial or yeast cell. In one preferred embodiment, the genetically engineered cell is a bacterial cell.

Host cells

Regarding the host cells, there are, in principle, no limitations; they may be eubacteria (gram-positive or gram-negative) or archaebacteria or fungi or even mammalian cells, as long as they allow genetic manipulation for insertion of a gene of interest and can be cultivated on a manufacturing scale. Preferably, the host cell has the property to allow cultivation to high cell densities.

In embodiments, the genetically engineered cell is a microorganism. The genetically engineered cell is preferably a microbial cell, such as a prokaryotic cell or eukaryotic cell. Appropriate microbial cells that may function as a host cell include bacterial cells, archaebacterial cells, algae cells and fungal cells.

The genetically engineered cell may be e.g., a bacterial or yeast cell. In one preferred embodiment, the genetically engineered cell is a bacterial cell.

Non-limiting examples of bacterial host cells that are suitable for recombinant industrial production of an HMO(s) according to the disclosure could be Erwinia herbicola (Pantoea agglomerans), Citrobacter freundii, Campylobacter sp, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this disclosure, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Streptococcus thermophiles and Proprionibacterium freudenreichii are also suitable bacterial species. Also included as part of this disclosure are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Streptomyces spp, Sporolactobacillus spp., Micromomospora spp., Micrococcus spp., Rhodococcus spp., and Pseudomonas (e.g., Pseudomonas fluorescens and Pseudomonas aeruginosa).

Non-limiting examples of fungal host cells that are suitable for recombinant industrial production of a heterologous product are e.g., yeast cells, such as Komagataella phaffii, Kluyveromyces lactis, Yarrowia lipolytica, Pichia pastoris, and Saccaromyces cerevisiae or filamentous fungi such as Aspargillus sp, Fusarium sp or Thricoderma sp, exemplary species are A. niger, A. nidulans, A. oryzae, F. solani, F. graminearum and T. reesei.

In one or more exemplary embodiments, the genetically engineered cell is selected from the group consisting of Escherichia coli, Corynebacterium glutamicum, lactobacillus lactis, Bacillus subtilis, Streptomyces lividans, Yarrowia lipolytica, Pichia pastoris and Saccharomyces cerevisiae.

In one or more exemplary embodiments, the genetically engineered cell is selected from the group consisting of of Escherichia Coli, Bacillus subtilis, lactobacillus lactis, Corynebacterium glutamicum, Yarrowia lipolytica, Pichia pastoris, and Saccharomyces cerevisiae.

In one or more exemplary embodiments, the genetically engineered cell is B. subtilis.

In one or more exemplary embodiments, the genetically engineered cell is S. Cerevisiae or P pastoris.

In one or more exemplary embodiments, the genetically engineered cell is Corynebacterium glutamicum.

In one or more exemplary embodiments, the genetically engineered cell is Escherichia coli.

In one or more exemplary embodiments, the disclosure relates to a genetically engineered cell, wherein the cell is derived from the E. coli K-12 strain or DE3.

### A Recombinant Nucleic Acid Sequence

The present disclosure relates to a genetically modified cell comprising a recombinant nucleic acid sequence encoding an enzyme with α-2,3-sialyltransferase and α-2,6-sialyltransferase activity, such as an enzyme selected from the group consisting of Chepa, Cinf1, Ccol2, Cjej1, Poral2 and CstII, wherein said cell produces Human Milk Oligosaccharides (HMO), in particular a mixture of sialylated HMOs, and preferably the mixture comprises or consists of 3′SL and 6′SL.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e., a promoter sequence.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence or other non-coding regulatory sequences.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The disclosure also relates to a nucleic acid construct comprising a coding nucleic sequence, i.e. recombinant DNA sequence of a gene of interest, e.g., a sialyltransferase gene, and a non-coding regulatory DNA sequence, e.g., a promoter DNA sequence, e.g., a recombinant promoter sequence derived from the promoter sequence of the lac operon or the glp operon, or a promoter sequence derived from another genomic promoter DNA sequence, or a synthetic promoter sequence, wherein the coding and promoter sequences are operably linked.

The term “operably linked” refers to a functional relationship between two or more nucleic acid (e.g., DNA) segments. It refers to the functional relationship of a transcriptional regulatory sequence to a transcribed sequence. E.g., a promoter sequence is operably linked to a coding sequence if it stimulates or modulates the transcription of the coding sequence in an appropriate host cell or other expression system.

Generally, promoter sequences that are operably linked to a transcribed sequence are physically contiguous to the transcribed sequence, i.e., they are cis-acting.

In one exemplified embodiment, the nucleic acid construct of the disclosure may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or expression of a gene/coding DNA sequence which may be included in the construct. Thus, in embodiments, the present disclosure relates to a nucleic acid construct comprising a recombinant nucleic acid sequence encoding a sialyltransferase, wherein said recombinant nucleic acid sequence is selected from the group consisting of nucleic acid sequences encoding Clari1, Neigon and Poral.

One embodiment of the disclosure is a nucleic acid construct comprising a recombinant nucleic acid sequence encoding a sialyltransferase, wherein said recombinant nucleic acid sequence is selected from the group consisting of a) Chepa comprising or consisting of the nucleic acid sequences of SEQ ID NO:7 or an nucleic acid sequence with at least 80%, such as at least 85%, such as at least 90%, such as at least 95%, or such as at least 99% identity to SEQ ID NO: 7; b) Cinf1 comprising or consisting of the nucleic acid sequences of SEQ ID NO: 8 or an nucleic acid sequence with at least 80%, such as at least 85%, such as at least 90%, such as at least 95%, or such as at least 99% identity to SEQ ID NO: 8; c) Ccol2 comprising or consisting of the nucleic acid sequence of SEQ ID NO: 9 or an nucleic acid sequence with at least 80%, such as at least 85%, such as at least 90%, such as at least 95%, or such as at least 99% identity to SEQ ID NO: 9, d) Cjej1 comprising or consisting of the nucleic acid sequence of SEQ ID NO: 10 or an nucleic acid sequence with at least 80%, such as at least 85%, such as at least 90%, such as at least 95%, or such as at least 99% identity to SEQ ID NO: 10, e) Poral2 comprising or consisting of the nucleic acid sequence of SEQ ID NO: 11 or an nucleic acid sequence with at least 80%, such as at least 85%, such as at least 90%, such as at least 95%, or such as at least 99% identity to SEQ ID NO: 1 f) CstII comprising or consisting of the nucleic acid sequence of SEQ ID NO: 12 or an nucleic acid sequence with at least 80%, such as at least 85%, such as at least 90%, such as at least 95%, or such as at least 99% identity to SEQ ID NO: 12. Preferably, the sialyltransferase encoding sequence is under the control of a promoter sequence selected from promotor sequences with a nucleic acid sequence as identified in Table 2.

TABLE 2
Selected promoter sequences

| | % Activity | | | Seq ID |
|---|---|---|---|---|
| Promoter name | relative to PglpF* | Strength | Reference | in appl. |
| PmglB_70UTR_SD8 | 291% | high | WO2020255054 | 19 |
| PmglB_70UTR_SD10 | 233-281% | high | WO2020255054 | 20 |
| PmglB_54UTR | 197% | high | WO2020255054 | 21 |
| Plac_70UTR | 182-220% | high | WO2019123324 | 22 |
| PmglB_70UTR_SD9 | 180-226% | high | WO2020255054 | 23 |
| PmglB_70UTR_SD4 | 153%-353% | high | WO2020255054 | 24 |
| PmglB_70UTR_SD5 | 146-152% | high | WO2020255054 | 25 |
| PglpF_SD4 | 140-161% | high | WO2019123324 | 26 |
| PmglB_70UTR_SD7 | 127-173% | high | WO2019123324 | 27 |
| PmglB_70UTR | 124-234% | high | WO2020255054 | 28 |
| PglpA_70UTR | 102-179% | high | WO2019123324 | 29 |
| PglpT_70UTR | 102-240% | high | WO2019123324 | 30 |
| PglpF | 100% | high | WO2019123324 | 31 |
| PglpF_SD10 | 88-96% | high | WO2019123324 | 17 |
| PglpF_SD5 | 82-91% | high | WO2019123324 | 32 |
| PglpF_SD8 | 81-82% | high | WO2019123324 | 33 |
| PmglB_16UTR | 78-171% | high | WO2019123324 | 34 |
| PglpF_SD9 | 73-93% | middle | WO2019123324 | 35 |
| PglpF_SD7 | 47-57% | middle | WO2019123324 | 36 |
| PglpF_SD6 | 46-47% | middle | WO2019123324 | 37 |
| PglpA_16UTR | 38-64% | middle | WO2019123324 | 38 |
| Plac_wt* | 15-28% | low | WO2019123324 | 18 |
| PglpF_SD3 | 9% | low | WO2019123324 | 39 |
| PglpF_SD1 | 5% | low | WO2019123324 | 40 |

*The promoter activity is assessed in the LacZ assay described below with the PglpF promoter run as positive reference in the same assay. To compare across assays the activity is calculated relative to the PglpF promoter, a range indicates results from multiple assays.

The promoter may be of heterologous origin, native to the genetically modified cell or it may be a recombinant promoter, combining heterologous and/or native elements.

One way to increase the production of a product may be to regulate the production of the desired enzyme activity used to produce the product, such as the glycosyltransferases or enzymes involved in the biosynthetic pathway of the glycosyl donor.

Increasing the promoter strength driving the expression of the desired enzyme may be one way of doing this. The strength of a promoter can be assessed using a lacZ enzyme assay where β-galactosidase activity is assayed as described previously (see e.g., Miller J. H. Experiments in molecular genetics, Cold spring Harbor Laboratory Press, NY, 1972). Briefly the cells are diluted in Z-buffer and permeabilized with sodium dodecyl sulfate (0.1%) and chloroform. The LacZ assays is performed at 30° C. Samples are preheated, the assay initiated by addition of 200 ul ortho-nitro-phenyl-ß-galactosidase (4 mg/ml) and stopped by addition of 500 ul of 1 M Na₂CO₃ when the sample had turned slightly yellow. The release of ortho-nitrophenol is subsequently determined as the change in optical density at 420 nm. The specific activities are reported in Miller Units (MU) [A420/(min*ml*A600)]. A regulatory element with an activity above 10,000 MU is considered strong and a regulatory element with an activity below 3,000 MU is considered weak, what is in between has intermediate strength. An example of a strong regulatory element is the PglpF promoter with an activity of approximately 14.000 MU and an example of a weak promoter is Plac which when induced with IPTG has an activity of approximately 2300 MU.

In one aspect of the disclosure, the expression of the recombinant nucleic acid sequence encoding an enzyme with α-2,3-sialyltransferase and α-2,6-sialyltransferase activity, is regulated by a promoter which confers an enhanced expression of said enzyme with dual-2,3-sialyltransferase and α-2,6-sialyltransferase, selected from the group of promoters consisting of Plac, PglpF, PmglB_70UTR PglpA_70UTR and PglpT_70UTR with a nucleic acid sequence according to SEQ ID Nos: 17, 28, 29, 30, respectively, and variants thereof (table 2).

In another aspect of the disclosure, the expression of the recombinant nucleic acid sequence encoding an enzyme with α-2,3-sialyltransferase and α-2,6-sialyltransferase activity, is regulated by a recombinant promoter which confers an enhanced expression of said enzyme with dual α-2,3-sialyltransferase and α-2,6-sialyltransferase activity.

Preferably, the nucleic acid construct comprising the recombinant nucleic acid encoding the α-2,3/6-sialyltransferase CstII further comprises a promoter sequence upstream of the nucleic acid encoding the α-2,3/6-sialyltransferase CstII, wherein the promoter sequence is selected from PglpF, PmglB_70UTR PglpA_70UTR and PglpT_70UTR with a nucleic acid sequence according to SEQ ID Nos: 17, 28, 29, 30, respectively, and variants thereof (table 2).

Additionally, constructs of the present disclosure may in addition comprise one or more nucleic acid sequences one or more MFS transporter such as a nucleic acid sequence of SEQ ID NO 14, 15 or 16 encoding Nec or YberC or Fred, respectively and one or more nucleic acid sequences encoding one or more sialic acid sugar nucleotide synthesis pathway enzymes such as a nucleic acid sequences of SEQ ID NO: 13 encoding the sialic acid sugar nucleotide synthesis pathway enzymes. In embodiments the expression of said nucleic acid sequences of the present disclosure is under control of a PglpF (SEQ ID NO: 17) or Plac (SEQ ID NO:18) promoter or PmglB_70UTR (SEQ ID NO: 28) or PlgpA_70UTR (Seq ID NO: 29) or PlgpT_70UTR (Seq ID NO: 30) or variants thereof such as promoters identified in Table 2. Further suitable variants of PglpF, PglpA, PglpT and PmglB promoter sequences are described in or WO2019/123324 and WO2020/255054 respectively (hereby incorporated by reference).

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2 (2): 137-49.); methods for genomic integration of nucleic acid sequences in which recombination is mediated by the Red recombinase function of the phage A or the RecE/RecT recombinase function of the Rac prophage (Murphy, J Bacteriol. (1998); 180 (8): 2063-7; Zhang et al., Nature Genetics (1998) 20:123-128 Muyrers et al., EMBO Rep. (2000) 1 (3): 239-243); methods based on Red/ET recombination (Wenzel et al., Chem Biol. (2005), 12 (3): 349-56.; Vetcher et al., Appl Environ Microbiol. (2005); 71 (4): 1829-35); or positive clones, i.e., clones that carry the expression cassette, can be selected e.g., by means of a marker gene, or loss or gain of gene function.

In one or more exemplary embodiments, the present disclosure relates to one or more recombinant nucleic acid sequences as illustrated in SEQ ID NO: 7, 8, 9, 10, 11 or 12.

In particular, the present disclosure relates to one or more of a recombinant nucleic acid sequence and/or to a functional homologue thereof having a sequence which is at least 70% identical to SEQ ID NO: 7, 8, 9, 10, 11 or 12, such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the disclosure) and a reference sequence (such as a prior art sequence) based on their pairwise alignment. For purposes of the present disclosure, the sequence identity between two amino acid sequences is determined using the Needleman-Wunsch algorithm (Needleman and Wunsch, 1970, J. Mo/. Biol. 48:443-453) as implemented in the Needle program of the EMBOSS package (EMBOSS: The European Molecular Biology Open Software Suite, Rice et al., 2000, Trends Genet. 16:276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the-nobrief option) is used as the percent identity and is calculated as follows: (Identical Residues×100)/(Length of Alignment-Total Number of Gaps in Alignment).

For purposes of the present disclosure, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16:276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the-nobrief option) is used as the percent identity and is calculated as follows: (Identical Deoxyribonucleotides×100)/(Length of Alignment−Total Number of Gaps in Alignment).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as at least 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in table 1 or a recombinant nucleic acid encoding any one of the sequences of table 4, should ideally be able to participate in the production of sialylated HMOs, in terms of increased HMO yield, export of HMO product out of the cell or import of substrate for the HMO production, such as lactose, improved purity/by-product formation, reduction in biomass formation, viability of the genetically engineered cell, robustness of the genetically engineered cell according to the disclosure, or reduction in consumables needed for the production.

### Use of a genetically modified cell

The disclosure also relates to any commercial use of the genetically modified cell(s) or the nucleic acid construct(s) disclosed herein, such as, but not limited to, in a method for producing a sialylated human milk oligosaccharide (HMO) and in particular in a method for producing a mixture of sialylated human milk oligosaccharides (HMOs).

In an exemplified embodiment, the genetically modified cell and/or the nucleic acid construct according to the disclosure is used in the manufacturing of HMOs.

In an exemplified embodiment, the genetically modified cell and/or the nucleic acid construct according to the disclosure is used in the manufacturing of sialylated HMO(s), wherein the sialylated HMOs are 3′SL and 6′SL.

In an exemplified embodiment, the genetically modified cell and/or the nucleic acid construct according to the disclosure is used in the manufacturing of a mixture of HMO(s) consisting of 3′SL and 6′SL.

In the present disclosure, the production of mixtures of 3′SL and 6′SL only requires the presence of a single sialyltransferase with dual activity.

### A Method for Producing Sialylated Human Milk Oligosaccharides (HMOs)

The present disclosure relates to a method for producing a mixture of sialylated human milk oligosaccharides (HMOs), said method comprises culturing a genetically modified cell according to the present disclosure. Example 1 of the present disclosure has identified the heterologous α-2,3/6-sialyltransferases Chepa, Cinf1, Ccol2, Cjej1, Poral2 and CstII (SEQ ID NO: 1, 2, 3, 4, 5 and 6 respectively), which, when expressed in a production strain, produce both 3′SL and 6′SL.

The present disclosure relates to a method for producing human milk oligosaccharides (HMOs), and in particular to a method for producing mixtures of 3′SL and 6′SL. The method of the present disclosure produces a mixture of sialylated human milk oligosaccharides (HMOs), wherein the molar content of each HMO in the mixture is above 20% of the total molar content of HMO produced by the method.

The present disclosure relates to a method for producing mixtures of 3′SL and 6′SL, wherein the molar ratio of 3′SL and 6′SL produced by the cell is between 25:75 and 80:20, such as 25:75, 30:70, 35:65, 40:60, 45:55, 50:50, 55:45, 60:40, 65:35, 70:30, 75:25 or such as 80:20. depending on the specific sialyltransferase.

When the method applies a genetically engineered cell that comprises a α-2,3/6-sialyltransferases selected from Chepa or Cinf1 or Ccol2 the 3′SL: 6′SL ratio is between 80:55and 75:60, specifically there is always at least 5%, such as 10% or 20% more 3′SL than 6′SL. When the method applies a genetically engineered cell that comprises α-2,3/6-sialyltransferases selected from Poral2 or Cjej1 the 3′SL: 6′SL ratio is between 20:80 and 30:70, specifically there is always at least 5%, such as 10% or 20% more 6′SL than 3′SL.

When the method applies a genetically engineered cell that comprises the α-2,3/6-sialyltransferases Chepa the 3′SL: 6′SL ratio is between 80:20 and 70:30, such as approximately 75:25.

When the method applies a genetically engineered cell that comprises the α-2,3/6-sialyltransferase Cinf1, the 3′SL: 6′SL ratio is between 70:30 and 60:40, such as approximately 65:35.

When the method applies a genetically engineered cell that comprises the α-2,3/6-sialyltransferase Ccol2 the 3′SL: 6′SL ratio is between 55:45 and 65:35, such as approximately 60:40.

When the method applies a genetically engineered cell that comprises the α-2,3/6-sialyltransferase Cjej1 the 3′SL: 6′SL ratio is between 25:75 and 40:60, such as approximately 35:65.

When the method applies a genetically engineered cell that comprises the α-2,3/6-sialyltransferase Poral2 the 3′SL: 6′SL ratio is between 25:75 and 40:60, such as approximately 35:65.

When the method applies a genetically engineered cell that comprises the α-2,3/6-sialyltransferase CstII the 3′SL: 6′SL ratio is approximately 50:50.

The present disclosure thus relates to a method for producing at least two sialylated human milk oligosaccharide (HMO), said method comprising culturing a genetically modified cell comprising a recombinant nucleic acid sequence encoding an enzyme with dual α-2,3-sialyltransferase and α-2,6-sialyltransferase activity wherein said cell is capable of producing at least 20% 3′SL and 20% 6′SL of the total molar HMO content produced by the cell.

One embodiment of the present disclosure relates to a method for producing at least two different sialylated human milk oligosaccharides (HMOs), said method comprising culturing a genetically modified cell comprising a recombinant nucleic acid sequence encoding an enzyme with dual α-2,3-sialyltransferase and α-2,6-sialyltransferase activity, wherein said enzyme is selected from the group consisting of:
a. Chepa comprising or consisting of the amino acid sequence of SEQ ID NO: 1 or an amino acid sequence with at least 80%, such as 85%, such as 90%, such as 95%, or such as 99% identity to SEQ ID NO: 1,
b. Cinf1 comprising or consisting of the amino acid sequence of SEQ ID NO: 2 or an amino acid sequence with at least 80%, such as 85%, such as 90%, such as 95%, or such as 99% identity to SEQ ID NO: 2,
c. Ccol2 comprising or consisting of the amino acid sequence of SEQ ID NO: 3 or an amino acid sequence with at least 80%, such as 85%, such as 90%, such as 95%, or such as 99% identity to SEQ ID NO: 3,
d. Cjej1 comprising or consisting of the amino acid sequence of SEQ ID NO: 4 or an amino acid sequence with at least 80%, such as 85%, such as 90%, such as 95%, or such as 99% identity to SEQ ID NO: 4,
e. Poral2 comprising or consisting of the amino acid sequence of SEQ ID NO: 5 or an amino acid sequence with at least 80%, such as 85%, such as 90%, such as 95%, or such as 99% identity to SEQ ID NO: 5, and
f. CstII comprising or consisting of the amino acid sequence of SEQ ID NO: 6 or an amino acid sequence with at least 80%, such as 85%, such as 90%, such as 95%, or such as 99% identity to SEQ ID NO: 6,
wherein the sialylated human milk oligosaccharides (HMOs) produced are 3′SL and 6′SL.

In a presently preferred method, said method comprises culturing a genetically modified cell comprising a recombinant nucleic acid sequence encoding an enzyme with dual α-2,3-sialyltransferase and α-2,6-sialyltransferase activity according to the disclosure, wherein said cell is capable of producing at least 20% 3′SL and at least 20% 6′SL of the total molar HMO content produced by the cell.

In one or more exemplary embodiments, the α-2,3/6-sialyltransferase of the present disclosure is under control of a PglpF, a Plac, a PmglB_70UTR, a PlgpA_70UTR or a PlgpT_70UTR promoter or variants thereof as disclosed in table 2. Thus, in an exemplary embodiment, the α-2,3/6-sialyltransferase of the present disclosure is under control of a PglpF promoter or a variant thereof as disclosed in table 2. In another exemplary embodiment, the α-2,3/6-sialyltransferase of the present disclosure is under control of a PmglB_70UTR promoter or a variant thereof as disclosed in table 2. Preferably, the α-2,3/6-sialyltransferase of the present disclosure is under the control of a recombinant promoter selected from the group consisting of SEQ ID NOs 17, 19, 20, 21, 22, 23, 24, 24, 26, 27, 28, 29, 30, 31, 32, 33, 34, 35 and 36.

Further genetic modifications can e.g., be selected from inclusion of additional glycosyltransferases and/or metabolic pathway engineering, and inclusion of MFS transporters, as described in the above sections, which the skilled person will know how to combine into a genetically modified cell capable of producing at least two sialylated HMO's.

The method particularly comprises culturing a genetically modified cell that produces at least two sialylated HMO, in particular the sialylated HMOs 3′SL and 6′SL. Preferably, 3′SL and 6′SL are the only HMOs produced they the method of the present disclosure.

The method comprising culturing a genetically modified cell that produces at least two sialylated HMOs and further comprises culturing said genetically engineered cell in in the presence of an energy source (carbon source) selected from the group consisting of glucose, sucrose, fructose, xylose, and glycerol.

In one aspect, the method according to the present disclosure produces at least two sialylated human milk oligosaccharide (HMO), such as 3′SL and 6′SL.

In one aspect, the method according to the present disclosure produces 3′SL and 6′SL.

In one aspect, the method according to the present disclosure produces a mixture of 3′SL and 6′SL with at least 20% of each in the mixture.

To enable the production of sialylated HMOs in the method according to the present disclosure, the genetically modified cell may comprise a biosynthetic pathway for making a sialic acid sugar nucleotide.

Thus, in methods of the present disclosure, the genetically modified cell comprises a biosynthetic pathway for making a sialic acid sugar nucleotide. Preferably, in methods of the present disclosure, the sialic acid sugar nucleotide is CMP-Neu5Ac. Thus, in methods of the present disclosure the sugar nucleotide pathway is expressed by the genetically modified cell, wherein the CMP-Neu5Ac pathway is encoded by the neuBCA operon from Campylobacter jejuni of SEQ ID NO: 13 In methods of the present disclosure, the sialic acid sugar nucleotide pathway is encoded from a high-copy plasmid bearing the neuBCA operon.

The method of the present disclosure comprises providing a glycosyl donor, which is synthesized separately by one or more genetically engineered cells and/or is exogenously added to the culture medium from an alternative source, alternatively sialic acid can be added during cultivation of the cell.

In a preferred embodiment of the method of the present disclosure further comprises providing an acceptor saccharide as substrate for the HMO formation, the acceptor saccharide comprising at least two monosaccharide units, which is exogenously added to the culture medium and/or has been produced by a separate microbial fermentation.

In one aspect, the method of the present disclosure comprises providing an acceptor saccharide comprising at least two monosaccharide units, which is exogenously added to the culture medium and/or has been produced by a separate microbial fermentation and which is selected form lactose, LNT-II and LNT, preferably lactose. In a preferred embodiment the substrate for HMO formation is lactose which is fed to the culture during the fermentation of the genetically engineered cell.

The sialylated human milk oligosaccharide (HMO) is retrieved from the culture, either from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence, preferably under control of a PglpF promoter, encoding an enzyme with α-2,3-sialyltransferase and α-2,6-sialyltransferase activity, wherein said enzyme is selected from the group consisting of: Chepa comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1, Cinf1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2, Ccol2 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3, Cjej1 comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4,Poral2 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5 and CstII comprising or consisting of the amino acid sequence of SEQ ID NO: 6 or an amino acid sequence with at least 80%, such as at least 85%, such as at least 90%, such as at least 95%, or such as at least 99% identity to SEQ ID NO: 6,
   ii. optionally a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Chepa comprising or consisting of the amino acid sequence of SEQ ID NO: 1 or an amino acid sequence with at least 80%, such as at least 85%, such as at least 90%, such as at least 95%, or such as at least 99% identity to SEQ ID NO: 1 and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Cinf1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2 and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Ccol2 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3 and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Cjej1 comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4 and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Poral2 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5 and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is CstII comprising or consisting of the amino acid sequence of SEQ ID NO: 6 or an amino acid sequence with at least 80%, such as at least 85%, such as at least 90%, such as at least 95%, or such as at least 99% identity to SEQ ID NO: 6, preferably under control of a PglpF or a PmglB_70UTR or a PlgpA_70UTR or a PlgpT_70UTR promoter and
   ii. a nucleic acid sequence encoding an MFS transporter, such as but not limited to Fred, Nec and/or YberC, preferably under control of a PglpF or Plac promoter, and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Chepa comprising or consisting of the amino acid sequence of SEQ ID NO: 1 or an amino acid sequence with at least 80%, such as at least 85%, such as 90%, such as at least 95%, or such as at least 99% identity to SEQ ID NO: 1 and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Cinf1 comprising or consisting of the amino acid sequence of SEQ ID NO: 2 or an amino acid sequence with at least 80%, such as at least 85%, such as at least 90%, such as at least 95%, or such as at least 99% identity to SEQ ID NO: 2 and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Ccol2 comprising or consisting of the amino acid sequence of SEQ ID NO: 3 or an amino acid sequence with at least 80%, such as at least 85%, such as at least 90%, such as at least 95%, or such as at least 99% identity to SEQ ID NO: 3 and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Cjej1 comprising or consisting of the amino acid sequence of SEQ ID NO: 4 or an amino acid sequence with at least 80%, such as at least 85%, such as at least 90%, such as at least 95%, or such as at least 99% identity to SEQ ID NO: 4 and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL and 6′SL, said method comprising:
a) obtaining a genetically modified cell comprising
   i. a recombinant nucleic acid sequence encoding an enzyme with α-2,3-sialyltransferase and α-2,6-sialyltransferase activity, wherein said enzyme is Poral2 comprising or consisting of the amino acid sequence of SEQ ID NO: 5 or an amino acid sequence with at least 80%, such as at least 85%, such as at least 90%, such as at least 95%, or such as at least 99% identity to SEQ ID NO: 5 and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

In particular, the present disclosure relates to a method for producing 3′SL, and 6′SL said method comprising:
a) obtaining a genetically modified cell comprising
   i. a recombinant nucleic acid sequence encoding an enzyme with a α-2,3-sialyltransferase and α-2,6-sialyltransferase activity, wherein said enzyme is CstII comprising or consisting of the amino acid sequence of SEQ ID NO: 6 or an amino acid sequence with at least 80%, such as at least 85%, such as at least 90%, such as at least 95%, or such as at least 99% identity to SEQ ID NO: 6 preferably under control of a PglpF or a PmglB_70UTR or a PlgpA_70UTR or a PlgpT_70UTR promoter and
b) culturing said genetically modified cell in a carbon-source containing culture medium and in the presence of lactose, and
c) producing said sialylated human milk oligosaccharides (HMOs), in particular 3′SL and 6′SL, by said genetically modified cell, and
d) retrieving the sialylated human milk oligosaccharides (HMOs), in particular 3′SL and 6′SL, from the culture medium and/or the genetically modified cell.

Culturing or fermenting (used interchangeably herein) in a controlled bioreactor typically comprises (a) a first phase of exponential cell growth in a culture medium ensured by a carbon-source, and (b) a second phase of cell growth in a culture medium run under carbon limitation, where the carbon-source is added continuously together with the acceptor oligosaccharide, such as lactose, allowing formation of the HMO product in this phase. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

The term “manufacturing” and “manufacturing scale” or “large-scale production” or “large-scale fermentation”, are used interchangeably and defines a fermentation with a minimum volume of 100 L, such as 1000 L, such as 10.000 L, such as 100.000 L, such as 200.000 L culture broth. Usually, a “manufacturing scale” process is defined by being capable of processing large volumes yielding amounts of the HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. The carbon-source can be selected from the group consisting of glucose, sucrose, fructose, xylose, and glycerol. In one or more exemplary embodiments, the culturing media is supplemented with one or more energy and carbon sources selected form the group containing glycerol, sucrose, and glucose.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015/197082 (hereby incorporated by reference).

After carrying out the method of this disclosure, the sialylated HMO produced can be collected from the cell culture or fermentation broth in a conventional manner.

### Retrieving/Harvesting

The sialylated human milk oligosaccharide (HMO) is retrieved from the culture medium and/or the genetically modified cell. In the present context, the term “retrieving” is used interchangeably with the term “harvesting”. Both “retrieving” and “harvesting” in the context relate to collecting the produced HMO(s) from the culture/broth following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation broth) include extraction thereof from the biomass (i.e., the production cells).

After recovery from fermentation, HMO(s) are available for further processing and purification.

The HMOs can be purified according to the procedures known in the art, e.g., such as described in WO2017/182965 or WO2017/152918, wherein the latter describes purification of sialylated HMOs. The purified HMOs can be used as nutraceuticals, pharmaceuticals, or for any other purpose, e.g., for research.

In embodiments the mixture of 3′SL and 6′SL is further purified from the recovery from the fermentation to produce at least 75%, such as at least 80%, such as at least 85%, such as at least 90%, such as at least 95% pure 3′SL and 6′SL.

At the end of culturing, the oligosaccharide as product can be accumulated both in the intrα-and the extracellular matrix.

The method according to the present disclosure comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose, and glycerol. In one or more exemplary embodiments, the culturing media is supplemented with one or more energy and carbon sources selected form the group containing glycerol, sucrose, and glucose.

### Manufactured Product

The term “manufactured product” according to the use of the genetically engineered cell or the nucleic acid construct refer to the one or more HMOs intended as the one or more product HMO(s). The various products are described above.

Advantageously, the methods disclosed herein provide both a decreased ratio of by-product to product and an increased overall yield of the product (and/or HMOs in total). This, less by-product formation in relation to product formation, facilitates an elevated product production and increases efficiency of both the production and product recovery process, providing superior manufacturing procedure of HMOs.

The manufactured product may be a powder, a composition, a suspension, or a gel comprising one or more HMOs.

### Sequences

The current application contains a sequence listing in text format and electronical format which is hereby incorporated by reference.

An overview of the SEQ ID NOs used in the present application can be found in table 1 (α-2,3-sialyltransferase protein sequences), 2 (promoter sequences) and 4 (α-2,3-sialyltransferase DNA sequences), additional sequences described in the application is the DNA sequence encoding the neuBCA operon from Campylobacter jejuni (SEQ ID NO: 13) and the MFS transporter sequences Nec (SEQ ID NO: 14), YberC (SEQ ID NO: 15) and Fred (SEQ ID NO: 16).

### EMBODIMENTS

Various embodiments of present disclosure are described in the following items.
1. A genetically modified cell comprising a recombinant nucleic acid sequence encoding an enzyme with dual dual α-2,3-sialyltransferase/α-2,6-sialyltransferase activity—sialyltransferase/α-2,6-sialyltransferase activity, wherein said cell is capable of producing at least 20% 3′SL and at least 20% 6′SL of the total molar HMO content produced by the cell.
2. The genetically modified cell according to item 1, wherein said enzyme is selected from the group consisting of:
   a. Chepa comprising or consisting of the amino acid sequence of SEQ ID NO: 1 or an amino acid sequence with at least 80%, such as at least 85%, such as at least 90%, such as at least 95% identity to SEQ ID NO: 1,
   b. Cinf1 comprising or consisting of the amino acid sequence of SEQ ID NO: 2 or an amino acid sequence with at least 80%, such as at least 85%, such as at least 90%, such as at least 95% identity to SEQ ID NO: 2,
   c. Ccol2 comprising or consisting of the amino acid sequence of SEQ ID NO: 3 or an amino acid sequence with at least 80%, such as at least 85%, such as at least 90%, such as at least 95% identity to SEQ ID NO: 3,
   d. Cjej1 comprising or consisting of the amino acid sequence of SEQ ID NO: 4 or an amino acid sequence with at least 80%, such as at least 85%, such as at least 90%, such as at least 95% identity to SEQ ID NO: 4,
   e. Poral2 comprising or consisting of the amino acid sequence of SEQ ID NO: 5 or an amino acid sequence with at least 80%, such as at least 85%, such as at least 90%, such as at least 95% identity to SEQ ID NO: 5, and
   f. CstII comprising or consisting of the amino acid sequence of SEQ ID NO: 6 or an amino acid sequence with at least 80%, such as at least 85%, such as at least 90%, such as at least 95% identity to SEQ ID NO: 6, wherein the expression of CstII is under the control of a promoter selected from the group consisting of PglpF, PmglB _70UTR or a PlgpA_70UTR or a PlgpT_70UTR and variants thereof.
3. The genetically modified cell according to any of items 1 or 2, wherein the cell only produces 3′SL and 6′SL.
4. The genetically modified cell according to any one of the preceding items, wherein the nucleic acid sequence encoding an enzyme with dual α-2,3-sialyltransferase/α-2,6-sialyltransferase activity is under the control of a promoter selected from the group consisting of PglpF, Plac, PmglB_70UTR, PlgpA_70UTR and PlgpT_70UTR with a nucleic acid sequence according to SEQ ID NOs 17, 18, 28, 29, 30, respectively or variants thereof, preferably the promoter is a strong promoter selected from the group consisting of SEQ ID NOs 17, 19, 20, 21, 22, 23, 24, 24, 26, 27, 28, 29, 30, 31, 32, 33, 34, 35 and 36.
5. The genetically modified cell according to any one of the preceding items, wherein the cell further comprises a nucleic acid sequence encoding an MFS transporter protein capable of exporting the sialylated HMO into the extracellular medium.
6. The genetically modified cell according to item 5, wherein the MFS transporter protein is the Fred (SEQ ID NO: 16), YberC (SEQ ID NO: 15) or Nec (SEQ ID NO: 14) protein or variants thereof.
7. The genetically modified cell according to any one of the preceding items, wherein the cell comprises a biosynthetic pathway for making a sialic acid sugar nucleotide.
8. The genetically modified cell according to item 7, wherein the sialic acid sugar nucleotide is CMP-Neu5Ac and said sialic acid sugar nucleotide pathway is encoded by the nucleic acid sequence encoding neuBCA from Campylobacter jejuni (SEQ ID NO: 13).
9. The genetically modified cell according to item 7 or 8, wherein the sialic acid sugar nucleotide pathway is encoded from a high-copy plasmid bearing the neuBCA operon.
10. The genetically modified cell according to any of the preceding items, wherein said modified cell is a microorganism.
11. The genetically modified cell according to any of the preceding items, wherein said modified cell is a bacterium or a fungus.
12. The genetically modified cell according to item 11, wherein said fungus is selected from a yeast cell of the genera Komagataella, Kluyveromyces, Yarrowia, Pichia, Saccaromyces, Schizosaccharomyces or Hansenula or from a filamentous fungous of the genera Aspargillus, Fusarium or Thricoderma.
13. The genetically modified cell according to item 11, wherein said bacterium is selected from the group consisting of Escherichia sp., Bacillus sp., Corynebacterium sp., lactobacillus sp. and Campylobacter sp.
14. The genetically modified cell according to item 13 wherein said bacterium is E. coli.
15. A method for producing at least two different sialylated human milk oligosaccharides (HMOs), said method comprising culturing a genetically modified cell according to any one of the preceding items, wherein said enzyme is selected from the group consisting of:
   a. Chepa comprising or consisting of the amino acid sequence of SEQ ID NO: 1 or an amino acid sequence with at least 80%, such as at least 85%, such as at least 90%, such as at least 95% identity to SEQ ID NO: 1,
   b. Cinf1 comprising or consisting of the amino acid sequence of SEQ ID NO: 2 or an amino acid sequence with at least 80%, such as at least 85%, such as at least 90%, such as at least 95% identity to SEQ ID NO: 2,
   c. Ccol2 comprising or consisting of the amino acid sequence of SEQ ID NO: 3 or an amino acid sequence with at least 80%, such as at least 85%, such as at least 90%, such as at least 95% identity to SEQ ID NO: 3,
   d. Cjej1 comprising or consisting of the amino acid sequence of SEQ ID NO: 4 or an amino acid sequence with at least 80%, such as at least 85%, such as at least 90%, such as at least 95% identity to SEQ ID NO: 4,
   e. Poral2 comprising or consisting of the amino acid sequence of SEQ ID NO: 5 or an amino acid sequence with at least 80%, such as at least 85%, such as at least 90%, such as at least 95% identity to SEQ ID NO: 5, and
   f. CstII comprising or consisting of the amino acid sequence of SEQ ID NO: 6 or an amino acid sequence with at least 80%, such as at least 85%, such as at least 90%, such as at least 95% identity to SEQ ID NO: 6, and wherein the sialylated human milk oligosaccharides (HMOs) produced are 3′SL and 6′SL, and said genetically modified cell optionally comprises at least one additional modification according to items 4 to 9.
16. The method according to item 15, wherein the molar ratio of the produced sialylated human milk oligosaccharides (HMOs) 3′SL: 6′SL is between 25:75 and 80:20.
17. The method according to item 15 or 16, wherein the molar ratio of the produced sialylated human milk oligosaccharides (HMOs) 3′SL: 6′S is:
   a. between 80:20 and 70:30, when the genetically engineered cell comprises the sialyl Transferase Chepa comprising or consisting of the amino acid sequence of SEQ ID NO: 1 or an amino acid sequence with at least 80%, such as at least 85%, such as at least 90%, such as at least 95% identity to SEQ ID NO: 1,
   b. between 70:30 to 60:40, when the genetically engineered cell comprises the sialyl transferase Cinf1 comprising or consisting of the amino acid sequence of SEQ ID NO: 2 or an amino acid sequence with at least 80%, such as at least 85%, such as at least 90%, such as at least 95% identity to SEQ ID NO: 2,
   c. between 55:45 and 65:35, when the genetically engineered cell comprises the sialyl transferase Ccol2 comprising or consisting of the amino acid sequence of SEQ ID NO: 3 or an amino acid sequence with at least 80%, such as at least 85%, such as at least 90%, such as at least 95% identity to SEQ ID NO: 3,
   d. between 30:70 and 40:60, when the genetically engineered cell comprises the sialyl transferase Cjej1 comprising or consisting of the amino acid sequence of SEQ ID NO: 4 or an amino acid sequence with at least 80%, such as at least 85%, such as at least 90%, such as at least 95% identity to SEQ ID NO: 4,
   e. between 25:75 and 40:60, when the genetically engineered cell comprises the sialyl transferase Poral2 comprising or consisting of the amino acid sequence of SEQ ID NO: 5 or an amino acid sequence with at least 80%, such as at least 85%, such as at least 90%, such as at least 95% identity to SEQ ID NO: 5 or
   f. approximately 50:50, when the genetically engineered cell comprises the sialyl transferase CstII comprising or consisting of the amino acid sequence of SEQ ID NO: 6 or an amino acid sequence with at least 80%, such as at least 85%, such as at least 90%, such as at least 95% identity to SEQ ID NO: 6.
18. The method according to any one of items 15 to 17, wherein the method comprises cultivating the genetically engineered cell in a culture medium which contains one or more carbohydrate source.
19. The method according to item 18, wherein the method comprises cultivating the genetically engineered cell in the presence of a carbon source selected from the group consisting of glucose, sucrose, fructose, xylose and glycerol.
20. The method according to item 15 to 19, wherein lactose is added during the cultivation of the genetically engineered cells as a substrate for the sialylated HMO formation.
21. The method according to any one of items 15 to 20, wherein the sialylated human milk oligosaccharides (HMOs) are retrieved from the culture medium and/or the genetically modified cell.
22. A nucleic acid construct comprising recombinant nucleic acid sequence encoding a sialyltransferase with dual α-2,3-sialyltransferase/α-2,6-sialyltransferase activity, wherein said recombinant nucleic acid sequence is selected from the group consisting of:
   a. Chepa comprising or consisting of the nucleic acid sequence of SEQ ID NO: 7 or a nucleic acid sequence with at least 80%, such as at least 85%, such as at least 90%, such as at least 95% identity to SEQ ID NO: 7,
   b. Cinf1 comprising or consisting of the nucleic acid sequence of SEQ ID NO: 8 or a nucleic acid sequence with at least 80%, such as at least 85%, such as at least 90%, such as at least 95% identity to SEQ ID NO: 8,
   c. Ccol2 comprising or consisting of the nucleic acid sequence of SEQ ID NO: 9 or a nucleic acid sequence with at least 80%, such as at least 85%, such as at least 90%, such as at least 95% identity to SEQ ID NO: 9,
   d. Cjej1 comprising or consisting of the nucleic acid sequence of SEQ ID NO: 10 or a nucleic acid sequence with at least 80%, such as at least 85%, such as at least 90%, such as at least 95% identity to SEQ ID NO: 10,
   e. Poral_2 comprising or consisting of the nucleic acid sequence of SEQ ID NO: 11 or a nucleic acid sequence with at least 80%, such as at least 85%, such as at least 90%, such as at least 95% identity to SEQ ID NO: 11, and
   f. CstII comprising or consisting of the nucleic acid sequence of SEQ ID NO: 12 or a nucleic acid sequence with at least 80%, such as at least 85%, such as at least 90%, such as at least 95% identity to SEQ ID NO: 12,
      wherein the sialyltransferase encoding sequence is under the control of a promoter sequence selected from the group consisting of PglpF, Plac, PmglB_70UTR or a PlgpA_70UTR or a PlgpT_70UTR (SEQ ID NOS: 17, 18, 28, 29, 30) and variants thereof.
23. A nucleic acid construct to item 22 for use in a host cell for producing at least two different sialylated HMOs.
24. A genetically modified cell according to any one of items item 1 to 14 for use in production of the sialylated human milk oligosaccharides (HMOs) 3′SL and 6′SL.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g.,, in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Enzymes

28 enzymes were collected following an in-silico selection approach that was based on protein BLAST searches using known α-2,3-sialyltransferases as queries and by exploiting information sources such as scientific articles or databases, e.g., the KEGG and CAZY databases.

TABLE 3
List of the enzymes tested in the framework of the present disclosure

| Enzyme | GenBank | Enzyme | |
|---|---|---|---|
| Name | ID | Length | Origin |
| Poral2 | WP_101774701.1 | 20 aa N-terminal | Pasteurella oralis |
| | | deletion | |
| Ccol2 | EAH6554614.1 | full length | Campylobacter coli |
| Cjej1 | EBD1936710.1 | full length | Campylobacter jejuni |
| Chepa | WP_066776435.1 | full length | Campylobacter hepaticus |
| Csub1 | WP_039664428.1 | full length | Campylobacter subantarcticus |
| Cstl | AAF13495.1 | 130 aa C-terminal | Campylobacter jejuni |
| | | deletion | |
| Clari1 | EGK8106227.1 | full length | Campylobacter lari |
| Cstll | AAF31771.1 | 32 aa C-terminal | Campylobacter jejuni |
| | | deletion | |
| Ccol | WP_075498955.1 | full length | Campylobacter coli |
| MhnN | WP_176810284.1 | full length | Mannheimia (multispecies) |
| Pmult | WP_005753497.1 | 24 aa N-terminal | Pasteurela multocida |
| | | deletion | |
| PM70 | AAK03258.1 | 31aa C-terminal | Pasteurella multocida subsp. |
| | | deletion | multocida str. Pm70 |
| Neigon | AAW89748.1 | 18 aa N-terminal | Neisseria gonorrhoeae FA 1090 |
| | | deletion | |
| Poral | WP_101774487.1 | full length | Pasteurella oralis |
| Cinf1 | WP_011272254.1 | full length | Haemophilus influenzae |
| PmN | WP_005726268.1 | full length | Pasteurella (multispecies) |
| Avi | WP_115249238.1 | 18 aa N-terminal | Avibacterium avium |
| | | deletion | |
| Nst | AAC44541.1 | 29 aa N-terminal | Neisseria meningitidis MC58 |
| | | deletion | |

### Strains

The strains (genetically engineered cells) constructed in the present application were based on Escherichia coli K-12 DH1with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186:2673-81 and Warming et al 2005 Nucleic Acids Res. 33 (4): e36) with specific selection marker genes and screening methods.

Codon optimized DNA sequences encoding individual α-2,3-sialyltransferases were genomically integrated into the MDO strain. Additionally, each strain was transformed with a high-copy plasmid bearing the neuBCA operon from Campylobacter jejuni (SEQ ID NO: 13) under the control of the Plac promoter. The neuBCA operon encodes all the enzymes required for the formation of an activated sialic acid sugar nucleotide (CMP-Neu5Ac). CMP-Neu5Ac acts as a donor for the intended glycosyltransferase reaction facilitated by the α-2,3-sialyltransferase under investigation, i.e., the transfer of sialic acid from the activated sugar CMP-Neu5Ac to the terminal galactose of lactose (acceptor) to form 3′SL.

The genotypes of the background strain (MDO), and the strains expressing enzymes with dual α-2,3-sialyltransferase and α-2,6-sialyltransferase activity capable of producing 3′SL and 6′SL are provided in Table 4.

TABLE 4
Genotypes of the strains, capable of producing 3′SL used in the present examples.

| Strain | | 2,3/6-ST cDNA |
|---|---|---|
| ref | Genotype | SEQ ID NO |
| MDO | F− λ− ΔendA1 ΔrecA1 ΔrelA1 ΔgyrA96 Δthi-1 glnV44 | |
| | hsdR17(rk−mK−) ΔlacZ wcaF::Plac ΔnanKETA ΔlacA ΔmelA | |
| | ΔwcaJ ΔmdoH | |
| Chepa | MDO Chepa_opt(PglpF), pBS-neuBCA(Plac)-amp | 7 |
| Cinf1 | MDO Cinf1_opt(PglpF), pBS-neuBCA(Plac)-amp | 8 |
| Ccol2 | MDO Ccol2_opt(PglpF), pBS-neuBCA(Plac)-amp | 9 |
| Poral2 | MDO Poral2_opt(PglpF), pBS-neuBCA(Plac)-amp | 10 |
| Cjej1 | MDO Cjej1_opt(PglpF), pBS-neuBCA(Plac)-amp | 11 |
| Cstll | MDO galK::Cstll_opt(PglpF) | 12 |

*2,3/6-ST is an abbreviation of α-2,3/6-sialyltransferase

### Deep Well Assay

The strains were screened in 96 deep well plates using a 4-day protocol. During the first 24 hours, precultures were grown to high densities and subsequently transferred to a medium that allowed induction of gene expression and product formation. More specifically, during day 1, fresh precultures were prepared using a basal minimal medium supplemented with magnesium sulphate, thiamine and glucose. The precultures were incubated for 24 hours at 34° C. and 1000 rpm shaking and then further transferred to a new basal minimal medium (BMM, pH 7,5) to start the main culture. The new BMM was supplemented with magnesium sulphate, thiamine, a bolus of 20% glucose solution (50 ul per 100 mL) and a bolus of 20% lactose solution (5 ml per 100 ml). Moreover, 50% sucrose solution was provided as carbon source, accompanied by the addition of sucrose hydrolase (invertase), so that glucose was released at a rate suitable for C-limited growth. IPTG (50 mg/ml) was added to induce gene expression and ampicillin antibiotic (100 mg/ml). The main cultures were incubated for 72 hours at 28° C. and 1000 rpm shaking.

### Example 1-In Vivo 3′SL and 6′SL Synthesis

Genetically modified cells expressing individual sialyltransferase enzymes were screened for their ability to produce the sialylated HMO 3′SL.

Genetically modified strains expressing the 18 individual sialyltransferases (table 3) were generated as described in the “Method” section. The cells were screened in a in a fed-batch deep well assay setup as described in the “Method” section. The molar content and ratio of individual HMOs produced by the strains was measured by HPLC.

When evaluating the results of the screening it was surprisingly found that 6 of the sialyltransferases, in addition to the 2,3-sialyltransferase activity that they were screened for, also possessed 2,6-sialyltransferase activity, since they produced both 3′SL and 6′SL expressing a single sialyltransferase.

Table 4 lists the genotype of the 6 strains that produced 3′SL and 6′SL.

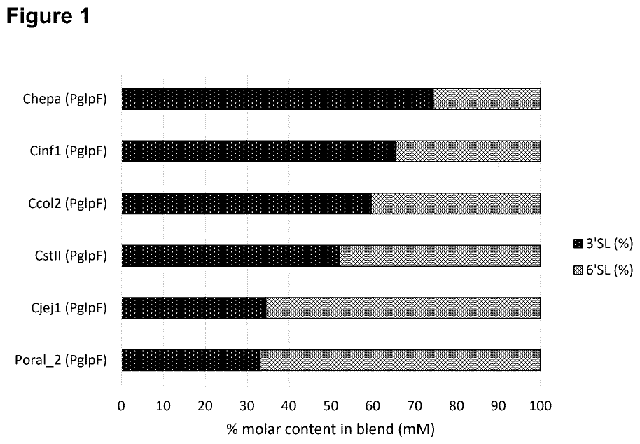

The results of the 3′SL/6′SL producing cells are shown in table 5 and illustrated in FIG. 1 as the molar ratio of 3′SL: 6′SL.

TABLE 5
Content of individual HMO's as % of total
HMO content produced by each strain.

| Strain ref. | 3′SL | 6′SL |
|---|---|---|
| Chepa | 74 | 26 |
| Cinf1 | 65 | 35 |
| Ccol2 | 60 | 40 |
| Cstll | 52 | 48 |
| Cjej1 | 35 | 65 |
| Poral2 | 33 | 67 |

From the data presented in table 5 it can be seen that there are the 6 enzymes (Chepa, Cinf1, Ccol2, Cjej1, Poral2 and CstII) that can form different molar ratios of 3′SL and 6′SL with a significant amount of both HMOs.

Chepa, Cinf1, Ccol2 produced at least 1.5 times more 3′SL than 6′SL while Cjej1 and Poral2 produced at least 1.8 times more 6′SL than 3′SL. CstII appeared to produce 3′SL and 6′SL in 10 an approximately 50:50 ratio. This shows that the molar ratio of the 3′SL and 6′SL produced by a genetically modified cell may be varied through the choice of specific enzymes.

1. A genetically modified cell comprising a recombinant nucleic acid sequence encoding an enzyme with dual α-2,3-sialyltransferase/α-2,6-sialyltransferase activity wherein the cell is capable of producing at least 20% 3′SL and at least 20% 6′SL of the total molar HMO content produced by the cell.

2. The genetically modified cell according to claim 1, wherein the enzyme is selected from the group consisting of:
a. Chepa comprising the amino acid sequence of SEQ ID NO: 1 or an amino acid sequence with at least 80%, identity to SEQ ID NO: 1,
b. Cinf1 comprising the amino acid sequence of SEQ ID NO: 2 or an amino acid sequence with at least 80%, identity to SEQ ID NO: 2,
c. Ccol2 comprising the amino acid sequence of SEQ ID NO: 3 or an amino acid sequence with at such as at least 90%, identity to SEQ ID NO: 3,
d. Cjej1 comprising the amino acid sequence of SEQ ID NO: 4 or an amino acid sequence with at least 80% identity to SEQ ID NO: 4,
e. Poral2 comprising the amino acid sequence of SEQ ID NO: 5 or an amino acid sequence with at least 80% identity to SEQ ID NO: 5, and
f. CstII comprising the amino acid sequence of SEQ ID NO: 6 or an amino acid sequence with at least 80% identity to SEQ ID NO: 6, wherein the expression of CstII is under the control of a promoter selected from the group consisting of PglpF, PmglB_70UTR, PlgpA_70UTR, and PlgpT_70UTR; or a variant thereof,
wherein the PglpF promoter comprises the nucleic acid sequence set forth in SEQ ID NO: 21, the PmglB_70UTR promoter comprises the nucleic acid sequence set forth in SEQ ID NO: 28, the PlgpA_70UTR promoter comprises the nucleic acid sequence set forth in SEQ ID NO: 29, and the PlgpT_70UTR promoter comprises the nucleic acid set forth in SEQ ID NO: 30.

3. The genetically modified cell according to claim 1, wherein the cell only produces the HMOs 3′SL and 6′SL.

4. The genetically modified cell according to claim 1, wherein the cell further comprises a nucleic acid sequence encoding an MFS transporter protein capable of exporting the sialylated HMO into the extracellular medium.

5. The genetically modified cell according to claim 4, wherein the MFS transporter protein is the Fred (SEQ ID NO: 16), YberC (SEQ ID NO: 15) or Nec (SEQ ID NO: 14) protein or a variants thereof.

6. The genetically modified cell according to claim 1, wherein the cell comprises a biosynthetic pathway for making a sialic acid sugar nucleotide.

7. The genetically modified cell according to claim 6, wherein the sialic acid sugar nucleotide is CMP-Neu5Ac and the sialic acid sugar nucleotide pathway is encoded by the nucleic acid sequence encoding neuBCA from Campylobacter jejuni (SEQ ID NO: 13).

8. The genetically modified cell according to claim 1, wherein the genetically modified cell is a microorganism.

9. The genetically modified cell according to claim 8, wherein the cell is selected from the group consisting of Escherichia Coli, Bacillus subtilis, lactobacillus lactis, Corynebacterium glutamicum, Yarrowia lipolytica, Pichia pastoris, and Saccharomyces cerevisiae.

10. The genetically modified cell according to claim 8, wherein said cell is E. coli.

11. A method for producing at least two different sialylated human milk oligosaccharides (HMOs), comprising culturing a genetically modified cell according to claim 1, wherein the enzyme is selected from the group consisting of:
a. Chepa comprising the amino acid sequence of SEQ ID NO: 1 or an amino acid sequence with at least 80% identity to SEQ ID NO: 1,
b Cinf1 comprising the amino acid sequence of SEQ ID NO: 2 or an amino acid sequence with at least 80% identity to SEQ ID NO: 2,
c. Ccol2 comprising the amino acid sequence of SEQ ID NO: 3 or an amino acid sequence with at least 80%, identity to SEQ ID NO: 3,
d. Cjej1 comprising the amino acid sequence of SEQ ID NO: 4 or an amino acid sequence with at least 80% identity to SEQ ID NO: 4,
e. Poral2 comprising the amino acid sequence of SEQ ID NO: 5 or an amino acid sequence with at least 80% identity to SEQ ID NO: 5, and
f. CstII comprising the amino acid sequence of SEQ ID NO: 6 or an amino acid sequence with at least 80%; identity to SEQ ID NO: 6, and
wherein the sialylated human milk oligosaccharides (HMOs) produced are 3′SL and 6′SL.

12. The method according to claim 11, wherein the molar ratio of the produced sialylated human milk oligosaccharides (HMOs) 3′SL: 6′SL is between 25:75 and 80:20.

13. The method according to claim 11, wherein the molar ratio of the produced sialylated human milk oligosaccharides (HMOs) 3′SL: 6′S is:
a. between 80:20 and 70:30, when the genetically engineered cell comprises the sialyl Transferase Chepa comprising the amino acid sequence of SEQ ID NO: 1 or an amino acid sequence with at least 80% identity to SEQ ID NO: 1,
b. between 70:30 to 60:40, when the genetically engineered cell comprises the sialyl transferase Cinf1 comprising the amino acid sequence of SEQ ID NO: 2 or an amino acid sequence with at least 80% identity to SEQ ID NO: 2,
c. between 55:45 and 65:35, when the genetically engineered cell comprises the sialyl transferase Ccol2 comprising the amino acid sequence of SEQ ID NO: 3 or an amino acid sequence with at least 80% identity to SEQ ID NO: 3,
d. between 30:70 and 40:60, when the genetically engineered cell comprises the sialyl transferase Cjej1 comprising the amino acid sequence of SEQ ID NO: 4 or an amino acid sequence with at least 80% identity to SEQ ID NO: 4,
e. between 25:75 and 40:60, when the genetically engineered cell comprises the sialyl transferase Poral2 comprising the amino acid sequence of SEQ ID NO: 5 or an amino acid sequence with at least 80% identity to SEQ ID NO: 5 or
f. approximately 50:50, when the genetically engineered cell comprises the sialyl transferase CstII comprising the amino acid sequence of SEQ ID NO: 6 or an amino acid sequence with at least 80% identity to SEQ ID NO: 6.

14. The method according to claim 11, wherein the method comprises cultivating the genetically engineered cell in a culture medium which contains one or more carbohydrate source.

15. The method according to claim 11, wherein lactose is added during the cultivation of the genetically engineered cells as a substrate for the sialylated HMO formation.

16. The method according to claim 11, wherein the sialylated human milk oligosaccharides (HMOs) are retrieved from the culture medium or the genetically modified cell.

17. A nucleic acid construct comprising recombinant nucleic acid sequence encoding a sialyltransferase with dual α-2,3-sialyltransferase/α-2,6-sialyltransferase activity, wherein said recombinant nucleic acid sequence is selected from the group consisting of:
a. Chepa comprising the nucleic acid sequence of SEQ ID NO: 7 or a nucleic acid sequence with at least 80% identity to SEQ ID NO: 7,
b. Cinf1 comprising of the nucleic acid sequence of SEQ ID NO: 8 or a nucleic acid sequence with at least 80%, identity to SEQ ID NO: 8,
c. Ccol2 comprising the nucleic acid sequence of SEQ ID NO: 9 or a nucleic acid sequence with at least 80% identity to SEQ ID NO: 9,
d. Cjej1 comprising the nucleic acid sequence of SEQ ID NO: 10 or a nucleic acid sequence with at least 80%, identity to SEQ ID NO: 10,
e. Poral_2 comprising the nucleic acid sequence of SEQ ID NO: 11 or a nucleic acid sequence with at least 80% identity to SEQ ID NO: 11, and
f. CstII comprising the nucleic acid sequence of SEQ ID NO: 12 or a nucleic acid sequence with at least 80% identity to SEQ ID NO: 12,
wherein the sialyltransferase encoding sequence is under the control of a promoter sequence selected from the group consisting of PglpF, Plac, PmglB_70UTR or a PlgpA_70UTR or a PlgpT_70UTR (SEQ ID NOs: 17, 18, 28, 29, 30) and variants thereof.

18. (canceled)

19. (canceled)

20. The genetically modified cell according to claim 1, wherein the variant of the PglpF, PmglB _70UTR, PlgpA_70UTR, or PlgpT_70UTR promoter comprises a promoter with the nucleic acid sequence set forth in SEQ ID NOs: 17, 19, 20, 22, 23, 24, 24, 26, 27, 31, 32, 33, 34, 35, 36, 37, 38, 39, or 40.

21. The genetically modified cell according to claim 1, wherein the enzyme is selected from the group consisting of:
a. Chepa comprising the amino acid sequence of SEQ ID NO: 1 or an amino acid sequence with at least 95% identity to SEQ ID NO: 1,
b. Cinf1 comprising the amino acid sequence of SEQ ID NO: 2 or an amino acid sequence with at least 95% identity to SEQ ID NO: 2,
c. Ccol2 comprising the amino acid sequence of SEQ ID NO: 3 or an amino acid sequence with at such as at least 95% identity to SEQ ID NO: 3,
d. Cjej1 comprising the amino acid sequence of SEQ ID NO: 4 or an amino acid sequence with at least 95% identity to SEQ ID NO: 4,
e. Poral2 comprising the amino acid sequence of SEQ ID NO: 5 or an amino acid sequence with at least 95% identity to SEQ ID NO: 5, and
f. CstII comprising the amino acid sequence of SEQ ID NO: 6 or an amino acid sequence with at least 95% identity to SEQ ID NO: 6.

22. The genetically modified cell according to claim 1, wherein the enzyme is selected from the group consisting of:
a. Chepa consisting of the amino acid sequence of SEQ ID NO: 1 or an amino acid sequence with at least 95% identity to SEQ ID NO: 1,
b. Cinf1 consisting of the amino acid sequence of SEQ ID NO: 2 or an amino acid sequence with at least 95% identity to SEQ ID NO: 2,
c. Ccol2 consisting of the amino acid sequence of SEQ ID NO: 3 or an amino acid sequence with at such as at least 95% identity to SEQ ID NO: 3,
d. Cjej1 consisting of the amino acid sequence of SEQ ID NO: 4 or an amino acid sequence with at least 95% identity to SEQ ID NO: 4,
e. Poral2 consisting of the amino acid sequence of SEQ ID NO: 5 or an amino acid sequence with at least 95% identity to SEQ ID NO: 5, and
f. CstII consisting of the amino acid sequence of SEQ ID NO: 6 or an amino acid sequence with at least 95% identity to SEQ ID NO: 6.
